(19) United States
(12) Patent Application Publication
(10) Pub. No.: US 2023/0236501 A1
(43) Pub. Date: Jul. 27, 2023

(54) RADIATION-SENSITIVE RESIN COMPOSITION, METHOD OF FORMING RESIST PATTERN, AND COMPOUND

(71) Applicant: JSR CORPORATION, Tokyo (JP)

(72) Inventors: Natsuko KINOSHITA, Tokyo (JP); Takuhiro TANIGUCHI, Tokyo (JP); Katsuaki NISHIKORI, Tokyo (JP); Kazuya KIRIYAMA, Tokyo (JP)

(73) Assignee: JSR CORPORATION, Tokyo (JP)

(21) Appl. No.: 18/100,031

(22) Filed: Jan. 23, 2023

(30) Foreign Application Priority Data
Jan. 25, 2022 (JP) 2022-009714
Nov. 30, 2022 (JP) 2022-192373

(51) Int. Cl. G03F 7/004; C07C 381/12; C07C 65/24; C07C 323/62; C07D 333/76; C07D 327/08; C07C 69/92; C08F 220/18; C08F 212/14

(52) U.S. Cl. CPC G03F 7/0045; C07C 381/12; C07C 65/24; C07C 323/62; C07D 333/76; C07D 327/08; C07C 69/92; C08F 220/1812; C08F 212/22; C08F 220/1811; C08F 220/1807; C07C 2601/08

(57) ABSTRACT

A radiation-sensitive resin composition includes: a polymer, solubility of which in a developer solution is capable of being altered by an action of an acid; a radiation-sensitive acid generator; and a compound represented by formula (1). Ar¹ represents a group obtained by removing (a+b+2) hydrogen atoms from an aromatic hydrocarbon ring having 6 to 30 ring atoms; R¹ represents a halogen atom or a monovalent organic group having 1 to 20 carbon atoms; L¹ represents a divalent linking group; R² represents a substituted or unsubstituted monovalent aromatic hydrocarbon group having 6 to 20 carbon atoms; a is an integer of 0 to 10, b is an integer of 1 to 10, wherein a sum of a and b is no greater than 10; and X⁺ represents a monovalent radiation-sensitive onium cation.

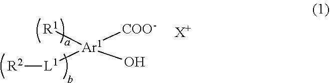

### CROSS-REFERENCE TO RELATED APPLICATIONS

The present application claims priority to Japanese Patent Application No. 2022-009714 filed Jan. 25, 2022, and to Japanese Patent Application No. 2022-192373 filed Nov. 30, 2022. The contents of these applications are incorporated herein by reference in their entirety.

### BACKGROUND OF THE INVENTION

### Field of the Invention

The present invention relates to a radiation-sensitive resin composition, a method of forming a resist pattern, and a compound.

### Description of the Related Art

A radiation-sensitive resin composition for use in microfabrication by lithography generates an acid at light-exposed regions upon an irradiation with a radioactive ray, e.g., an electromagnetic wave such as a far ultraviolet ray such as an ArF excimer laser beam (wavelength of 193 nm), a KrF excimer laser beam (wavelength of 248 nm), etc. or an extreme ultraviolet ray (EUV) (wavelength of 13.5 nm), or a charged particle ray such as an electron beam. A chemical reaction in which the acid serves as a catalyst causes a difference between the light-exposed regions and light-unexposed regions in rates of dissolution in a developer solution, whereby a resist pattern is formed on a substrate.

Such radiation-sensitive compositions are required not only to have favorable sensitivity to exposure light such as an extreme ultraviolet ray and an electron beam, but also to result in superiority in terms of CDU (Critical Dimension Uniformity) performance, resolution, and the like.

Types, molecular structures, and the like of polymers, acid generating agents, and other components which may be used in radiation-sensitive resin compositions have been investigated to meet these requirements, and combinations thereof have been further investigated in detail (see Japanese Unexamined Patent Applications, Publication Nos. 2010-134279, 2014-224984, and 2016-047815).

### SUMMARY OF THE INVENTION

According to an aspect of the present invention, a radiation-sensitive resin composition includes: a polymer, solubility of which in a developer solution is capable of being altered by an action of an acid; a radiation-sensitive acid generator; and a compound represented by formula (1).

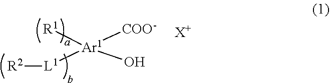

In the formula (1), Ar¹ represents a group obtained by removing (a+b+2) hydrogen atoms from an aromatic hydrocarbon ring having 6 to 30 ring atoms; R¹ represents a halogen atom or a monovalent organic group having 1 to 20 carbon atoms; L¹ represents a divalent linking group; R² represents a substituted or unsubstituted monovalent aromatic hydrocarbon group having 6 to 20 carbon atoms; a is an integer of 0 to 10; b is an integer of 1 to 10, wherein a sum of a and b is no greater than 10, and wherein in a case in which a is no less than 2, a plurality of R¹s are identical or different from each other, and wherein in a case in which b is no less than 2, a plurality of L¹s are identical or different from each other, and a plurality of R₂s are identical or different from each other; and X⁺ represents a monovalent radiation-sensitive onium cation.

According to another aspect of the present invention, a method of forming a resist pattern includes: forming a resist film directly or indirectly on a substrate by applying the above-described radiation-sensitive resin composition; exposing the resist film; and developing the resist film exposed.

According to a further aspect of the present invention, a compound is represented by formula (1).

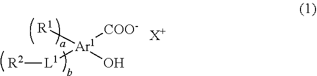

In the formula (1), Ar¹ represents a group obtained by removing (a+b+2) hydrogen atoms from an aromatic hydrocarbon ring having 6 to 30 ring atoms; R represents a halogen atom or a monovalent organic group having 1 to 20 carbon atoms; L¹ represents a divalent linking group; R² represents a substituted or unsubstituted monovalent aromatic hydrocarbon group having 6 to 20 carbon atoms; a is an integer of 0 to 10; b is an integer of 1 to 10, wherein a sum of a and b is no greater than 10, and wherein in a case in which a is no less than 2, a plurality of R¹s are identical or different from each other, and wherein in a case in which b is no less than 2, a plurality of Lis are identical or different from each other, and a plurality of R₂s are identical or different from each other; and X⁺ represents a monovalent radiation-sensitive onium cation.

### DESCRIPTION OF THE EMBODIMENTS

As used herein, the words “a” and “an” and the like carry the meaning of “one or more.” When an amount, concentration, or other value or parameter is given as a range, and/or its description includes a list of upper and lower values, this is to be understood as specifically disclosing all integers and fractions within the given range, and all ranges formed from any pair of any upper and lower values, regardless of whether subranges are separately disclosed. Where a range of numerical values is recited herein, unless otherwise stated, the range is intended to include the endpoints thereof, as well as all integers and fractions within the range. As an example, a stated range of 1-10 fully describes and includes the independent subrange 3.4-7.2 as does the following list of values: 1, 4, 6, 10.

Along with further miniaturization of resist patterns, required levels for the aforementioned types of performance are further elevated, and there are demands for radiation-sensitive resin compositions capable of meeting the requirements described above.

According to one embodiment of the invention, a radiation-sensitive resin composition contains: a polymer, solubility of which in a developer solution is capable of being altered by an action of an acid (hereinafter, may be also referred to as “(A) polymer” or “polymer (A)”); a radiation-sensitive acid generator (hereinafter, may be also referred to as “(C) acid generator” or “acid generator (C)”); and a compound represented by the following formula (1) (hereinafter, may be also referred to as “(D) compound” or “compound (D)”).

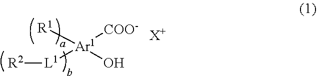

In the formula (1),

Ar¹ represents a group obtained by removing (a+b+2) hydrogen atoms from an aromatic hydrocarbon ring having 6 to 30 ring atoms;

R¹ represents a halogen atom or a monovalent organic group having 1 to 20 carbon atoms;

L¹ represents a divalent linking group;

R² represents a substituted or unsubstituted monovalent aromatic hydrocarbon group having 6 to 20 carbon atoms;
a is an integer of 0 to 10; b is an integer of 1 to 10, wherein a sum of a and b is no greater than 10, and wherein in a case in which a is no less than 2, a plurality of R¹s are identical or different from each other, and wherein in a case in which b is no less than 2, a plurality of L¹s are identical or different from each other, and a plurality of R²s are identical or different from each other; and

X⁺ represents a monovalent radiation-sensitive onium cation.

According to an other embodiment, a method of forming a resist pattern includes: applying the radiation-sensitive resin composition of the one embodiment of the present invention directly or indirectly on a substrate; exposing a resist film formed by the applying; and developing the resist film exposed.

Still another embodiment of the invention is the compound (D).

The radiation-sensitive resin composition and the method of forming a resist pattern of the embodiments of the present invention enable formation of a resist pattern with favorable sensitivity to exposure light and superiority in terms of the CDU performance and the resolution. The compound of the present embodiment can be suitably used as a component of the radiation-sensitive resin composition of the one embodiment of the present invention. Therefore, these can be suitably used for working processes of semiconductor devices, and the like, in which microfabrication is expected to be further in progress hereafter.

Hereinafter, the radiation-sensitive resin composition, the method of forming a resist pattern, and the compound of embodiments of the present invention will be described in detail.

Radiation-Sensitive Resin Composition

The radiation-sensitive composition of one embodiment of the present invention contains the polymer (A), the acid generator (C), and the compound (D). The radiation-sensitive resin composition typically contains an organic solvent (hereinafter, may be also referred to as “(E) organic solvent” or “organic solvent (E)”). The radiation-sensitive composition may contain, as a favorable component, a polymer (hereinafter, may be also referred to as “(B) polymer” or “polymer (B)”) having a percentage content of fluorine atoms greater than that of the polymer (A). The radiation-sensitive resin composition may contain, within a range not leading to impairment of the effects of the present invention, other optional component(s).

Due to the polymer (A), the acid generator (C), and the compound (D) being contained, the radiation-sensitive resin composition enables formation of a resist pattern with favorable sensitivity to exposure light and superiority in terms of the CDU performance and the resolution. Although not necessarily clarified and without wishing to be bound by any theory, the reason for achieving the aforementioned effects by the radiation-sensitive resin composition due to involving such a constitution may be presumed, for example, as in the following. Due to the compound (D) having a bulky structure, it is considered that diffusion lengths of an acid generated by exposure can be appropriately shortened, and as a result, the sensitivity to exposure light, the CDU performance, and the resolution can be improved.

The radiation-sensitive resin composition can be prepared by, for example, mixing the polymer (A), the acid generator (C), and the compound (D), as well as the polymer (B), the organic solvent (E), the other optional component(s), and the like, which are added as needed, in a certain ratio, and preferably filtering a thus resulting mixture through a membrane filter having a pore size of no greater than 0.20 m.

Each component contained in the radiation-sensitive resin composition is described below.

(A) Polymer

The polymer (A) is a polymer, solubility of which in a developer solution is capable of being altered by an action of an acid. In general, due to the polymer (A) having an acid-labile group, the property of altering the solubility in a developer solution by an action of an acid is exhibited. Therefore, the polymer (A) preferably has a structural unit (hereinafter, may be also referred to as “structural unit (I)”) that includes an acid-labile group. The radiation-sensitive resin composition may contain one type, or two or more types of the polymer (A).

It is preferred that the polymer (A) further has a structural unit (hereinafter, may be also referred to as “structural unit (II)”) that includes a phenolic hydroxy group. The polymer (A) may further have other structural unit(s) (hereinafter, may be also referred to merely as “other structural unit(s)”), aside from the structural units (I) and (II). The polymer (A) can have one type, or two or more types of each of the structural units.

The lower limit of the proportion of the polymer (A) contained in the radiation-sensitive resin composition is, with respect to total components other than the organic solvent (E) contained in the radiation-sensitive resin composition, preferably 50% by mass, more preferably 70% by mass, and still more preferably 80% by mass. The upper limit of the proportion is preferably 99% by mass, and more preferably 95% by mass.

The lower limit of a polystyrene-equivalent weight average molecular weight (Mw) of the polymer (A) as determined by gel permeation chromatography (GPC) is preferably 1,000, more preferably 3,000, and still more preferably 4,000. The upper limit of the Mw is preferably 50,000, more preferably 30,000, still more preferably 20,000, even further preferably 15,000, and particularly preferably 10,000. When the Mw of the polymer (A) falls within the above range, coating characteristics of the radiation-sensitive resin composition may be improved. The Mw of the polymer (A) can be adjusted by, for example, regulating the type, the amount, and the like of a polymerization initiator used in synthesis of the same.

The upper limit of a ratio (hereinafter may be also referred to as “dispersity index” or “Mw/Mn”) of the Mw to a polystyrene-equivalent number average molecular weight (Mn) of the polymer (A) as determined by GPC is preferably 2.5, more preferably 2.0, and still more preferably 1.7. The lower limit of the ratio is typically 1.0, preferably 1.1, more preferably 1.2, and still more preferably 1.3.

Method for Measuring Mw and Mn

As referred to herein, the Mw and Mn of the polymer are values measured by using gel permeation chromatography (GPC) under the following conditions.

GPC columns: “G2000 HXL”×2, “G3000 HXL”×1, and “G4000 HXL”×1, available from Tosoh Corporation

column temperature: 40° C.

elution solvent: tetrahydrofuran

flow rate: 1.0 mL/min

sample concentration: 1.0% by mass

amount of injected sample: 100 uL

detector: differential refractometer

standard substance: mono-dispersed polystyrene

The polymer (A) can be synthesized by, for example, polymerizing a monomer that gives each structural unit in accordance with a well-known procedure.

Each structural unit contained in the polymer (A) is described below.

Structural Unit (I)

The structural unit (I) is a structural unit that includes an acid-labile group. The “acid-labile group” as referred to herein means a group that substitutes for a hydrogen atom in a carboxy group, a hydroxy group, or the like, and is capable of being dissociated by an action of an acid to give a carboxy group, a hydroxy group, or the like. The acid-labile group is dissociated by an action of the acid generated from the acid generator (C), etc. upon exposure, whereby a difference is generated in the solubility of the polymer (A) in the developer solution, between light-exposed regions and light-unexposed regions, and thus forming a resist pattern is enabled. The polymer (A) may have one type, or two or more types of the structural unit (I).

Examples of the structural unit (I) include structural units represented by the following formulae (2-1) to (2-2), and the like.

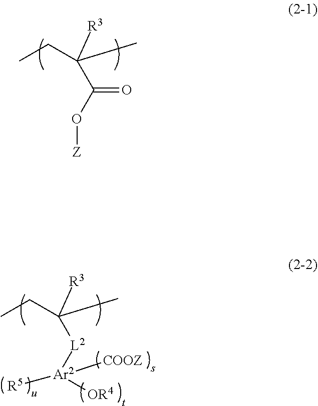

In the above formula (2-1) and (2-2), R³ represents a hydrogen atom, a fluorine atom, a methyl group, or a trifluoromethyl group; and Z represents an acid-labile group (hereinafter, may be also referred to as “acid-labile group (Z)”).

In the above formula (2-2),

L² represents a single bond, —COO—, —CONH—, or —O—;

Ar² represents a group obtained by removing (s+t+u+1) hydrogen atoms from an aromatic hydrocarbon ring having 6 to 20 ring atoms;

s is an integer of 0 to 10; t is an integer of 0 to 10, wherein a sum of s and t is an integer of 1 to 10, and wherein in a case in which s is no less than 2, a plurality of Zs are identical or different from each other, in a case in which s is no less than 1, R⁴ represents a hydrogen atom or a monovalent organic group having 1 to 20 carbon atoms, and wherein in a case in which t is no less than 2, a plurality of R⁴s are identical or different from each other, in a case in which s is 0 and t is 1, R⁴ represents an acid-labile group, and wherein in a case in which s is 0 and t is no less than 2, at least one of a plurality of R⁴s represents an acid-labile group;

R⁵ represents a halogen atom or a monovalent organic group having 1 to 20 carbon atoms; and

u is an integer of 0 to 10, wherein in a case in which u is no less than 2, a plurality of R⁵s are identical or different from each other, and wherein a sum of s, t, and u is no greater than 10.

The number of “ring atoms” as referred to herein means the number of atoms constituting a ring structure, and in the case of a polycyclic ring, the number of “ring atoms” means the number of atoms constituting the polycyclic ring. The “aromatic ring” encompasses an “aromatic hydrocarbon ring” and an “aromatic heterocyclic ring”. The “aromatic ring” encompasses a “monocyclic aromatic ring” and a “polycyclic aromatic ring”. The “polycyclic aromatic ring” may encompass a fused polycyclic rings in which two rings have two shared atoms, as well as a ring-assembled polycyclic ring in which two rings are connected by a single bond without having any shared atom.

The number of “carbon atoms” means the number of carbon atoms constituting a group. The “organic group” means a group having at least one carbon atom. The “hydrocarbon group” encompasses a chain hydrocarbon group, an alicyclic hydrocarbon group, and an aromatic hydrocarbon group. The “hydrocarbon group” may be either a saturated hydrocarbon group or an unsaturated hydrocarbon group. The “chain hydrocarbon group” as referred to herein means a hydrocarbon group not having a ring structure but being constituted with only a chain structure, and may be exemplified by both a linear hydrocarbon group and a branched hydrocarbon group. The “alicyclic hydrocarbon group” as referred to herein means a hydrocarbon group having, as a ring structure, not an aromatic ring but an aliphatic ring alone, and may be exemplified by both a monocyclic alicyclic hydrocarbon group and a polycyclic alicyclic hydrocarbon group. With regard to this, it is not necessary for the alicyclic hydrocarbon group to be constituted with only an aliphatic ring; it may have a chain structure in a part thereof. The “aromatic hydrocarbon group” as referred to herein means a hydrocarbon group that includes an aromatic ring as a ring structure. With regard to this, it is not necessary for the aromatic hydrocarbon group to be constituted with only an aromatic ring, and it may have a chain structure or an aliphatic ring in a part thereof. The “aliphatic hydrocarbon group” as referred to herein means a chain hydrocarbon group and an alicyclic hydrocarbon group.

R³ represents, in light of a degree of copolymerization of a monomer that gives a structural unit (I), preferably a hydrogen atom or a methyl group, and more preferably a hydrogen atom.

L² represents preferably a single bond.

Examples of the aromatic hydrocarbon ring having 6 to 20 ring atoms that gives Ar² include: a benzene ring; fused polycyclic aromatic hydrocarbon rings such as a naphthalene ring, a phenanthrene ring, an anthracene ring, and a fluorene ring; ring-assembled aromatic hydrocarbon rings such as a biphenyl ring, a terphenyl ring, a binaphthalene ring, and a phenylnaphthalene ring; and the like.

The aromatic hydrocarbon ring having 6 to 20 ring atoms that gives Ar² is preferably a benzene ring.

s is preferably 0 to 3, more preferably 0 to 2, and still more preferably 1.

The monovalent organic group having 1 to 20 carbon atoms which may be represented by R⁴ in a case in which s is no less than 1 is exemplified by: a monovalent hydrocarbon group having 1 to 20 carbon atoms; a group (α) including a divalent heteroatom-containing group between two adjacent carbon atoms of the monovalent hydrocarbon group; a group (β) obtained by substituting with a monovalent heteroatom-containing group, a part or all of hydrogen atoms included in the monovalent hydrocarbon group or the group (α); a group (γ) in which the monovalent hydrocarbon group, the group (α), or the group (β) is combined with a divalent heteroatom-containing group; and the like. Alternatively, the organic group in this case may be an acid-labile group (Z) described later.

The monovalent hydrocarbon group having 1 to 20 carbon atoms may be exemplified by a monovalent chain hydrocarbon group having 1 to 20 carbon atoms, a monovalent alicyclic hydrocarbon group having 3 to 20 carbon atoms, a monovalent aromatic hydrocarbon group having 6 to 20 carbon atoms, and the like.

Examples of the monovalent chain hydrocarbon group having 1 to 20 carbon atoms include: alkyl groups such as a methyl group, an ethyl group, an n-propyl group, and an i-propyl group; alkenyl groups such as an ethenyl group, a propenyl group, and a butenyl group; alkynyl groups such as an ethynyl group, a propynyl group, and a butynyl group; and the like

Examples of the monovalent alicyclic hydrocarbon group having 3 to 20 carbon atoms include: monocyclic alicyclic saturated hydrocarbon groups such as a cyclopentyl group and a cyclohexyl group; polycyclic alicyclic saturated hydrocarbon groups such as a norbornyl group, an adamantyl group, a tricyclodecyl group, and a tetracyclododecyl group; monocyclic alicyclic unsaturated hydrocarbon groups such as a cyclopentenyl group and a cyclohexenyl group; polycyclic alicyclic unsaturated hydrocarbon groups such as a norbornenyl group, a tricyclodecenyl group, and a tetracyclododecenyl group; and the like.

Examples of the monovalent aromatic hydrocarbon group having 6 to 20 carbon atoms include: aryl groups such as a phenyl group, a tolyl group, a xylyl group, a naphthyl group, and an anthryl group; aralkyl groups such as a benzyl group, a phenethyl group, a naphthylmethyl group, and an anthrylmethyl group; and the like.

The heteroatom that may constitute the monovalent or divalent heteroatom-containing group is exemplified by an oxygen atom, a nitrogen atom, a sulfur atom, a phosphorus atom, a silicon atom, a halogen atom, and the like. The halogen atom may be a fluorine atom, a chlorine atom, a bromine atom, or an iodine atom.

Examples of the divalent heteroatom-containing group include —O—, —CO—, —S—, —CS—, —NR′—, groups in which at least two of the aforementioned groups are combined (for example, —COO—, —CONR′—, etc.), and the like, wherein R′ represents a hydrogen atom or a monovalent hydrocarbon group having 1 to 10 carbon atoms. Examples of the monovalent hydrocarbon group having 1 to 10 carbon atoms which may be represented by R′ include, among the groups exemplified as the “monovalent hydrocarbon group having 1 to 20 carbon atoms,” groups having 1 to 10 carbon atoms, and the like.

In the case in which s is no less than 1, R⁴ represents preferably the monovalent organic group having 1 to 20 carbon atoms, more preferably a group in which the monovalent hydrocarbon group having 1 to 20 carbon atoms is combined with the divalent heteroatom-containing group, still more preferably a group in which the monovalent chain hydrocarbon group having 1 to 20 carbon atoms is combined with —CO—, and even further preferably an acyl group.

In the case in which s is 0, R⁴ is exemplified by a group similar to the acid-labile group (Z) described later.

t is preferably 0 to 2, and more preferably 1.

Examples of the monovalent organic group having 1 to 20 carbon atoms which may be represented by R⁵ include groups similar to the groups exemplified as the monovalent organic group having 1 to 20 carbon atoms which may be represented by R⁴, and the like.

The halogen atom which may be represented by R⁵ is a fluorine atom, a chlorine atom, a bromine atom, or an iodine atom.

u is preferably 0 or 1, and more preferably 0.

Acid-Labile Group (Z)

The acid-labile group (Z) is a group that substitutes for a hydrogen atom in the carboxy group, and that is dissociated by an action of an acid to give a carboxy group. Examples of the acid-labile group (Z) include groups represented by the following formulae (3-1) to (3-2), and the like.

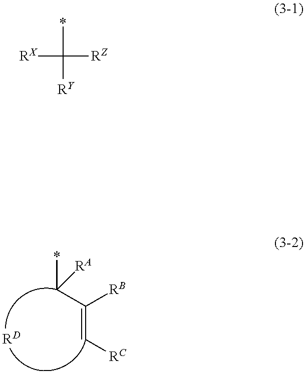

In the above formula (3-1), R^{X} represents a substituted or unsubstituted monovalent hydrocarbon group having 1 to 20 carbon atoms; R^{Y} and R^{Z} each independently represent a monovalent hydrocarbon group having 1 to 20 carbon atoms, or R^{Y} and R^{Z} taken together represent a saturated aliphatic ring having 3 to 20 ring atoms together with the carbon atom to which R^{Y} and R^{Z} bond.

In the above formula (3-2), R^{A} represents a hydrogen atom; R^{B} and R^{C} each independently represent a hydrogen atom or a monovalent hydrocarbon group having 1 to 20 carbon atoms; and R^{D} represents a divalent hydrocarbon group having 1 to 20 carbon atoms that constitutes the unsaturated aliphatic ring having 4 to 20 ring atoms together with carbon atoms to which R^{A}, R^{B}, and R^{C} bond, respectively.

Examples of the monovalent hydrocarbon group having 1 to 20 carbon atoms which may be represented by R^{X}, R^{Y}, R^{Z}, R^{B}, or R^{C} include, among the monovalent organic groups having 1 to 20 carbon atoms which may be represented by R⁴ in the above formula (2-2), groups similar to the groups exemplified as the monovalent hydrocarbon group having 1 to 20 carbon atoms, and the like.

Examples of the substituent which may be incorporated in the hydrocarbon group represented by R^{X} include halogen atoms such as a fluorine atom and an iodine atom, a hydroxy group, a carboxy group, a cyano group, a nitro group, an alkoxy group, an alkoxycarbonyl group, an alkoxycarbonyloxy group, an acyl group, an acyloxy group, and the like.

Examples of the saturated aliphatic ring having 3 to 20 ring atoms which may be represented by R^{Y} and R^{Z}, taken together, together with the carbon atom to which R^{Y} and R^{Z} bond include: monocyclic saturated aliphatic rings such as a cyclopropane ring, a cyclobutane ring, a cyclopentane ring, and a cyclohexane ring; polycyclic saturated aliphatic rings such as a norbornane ring and an adamantane ring; and the like.

Examples of the divalent hydrocarbon group having 1 to 20 carbon atoms represented by R^{D} include, among the monovalent organic groups having 1 to 20 carbon atoms which may be represented by R⁴ in the above formula (2-2), groups obtained by removing one hydrogen atom from the groups exemplified as the monovalent hydrocarbon group having 1 to 20 carbon atoms, and the like.

Examples of the unsaturated aliphatic ring having 4 to 20 ring atoms constituted from R^{D} together with three carbon atoms to which R^{A}, R^{B}, and R^{C} bond, respectively, include: monocyclic unsaturated aliphatic rings such as a cyclobutene ring, a cyclopentene ring, and a cyclohexene ring; polycyclic unsaturated aliphatic rings such as a norbornene ring; and the like.

R^{X} represents preferably a substituted or unsubstituted chain hydrocarbon group or a substituted or unsubstituted aromatic hydrocarbon group, more preferably an unsubstituted chain hydrocarbon group or an unsubstituted aromatic hydrocarbon group, still more preferably an unsubstituted alkyl group or an unsubstituted aryl group, and even more preferably a methyl group, an ethyl group, or a phenyl group.

R^{Y} represents preferably a chain hydrocarbon group or an alicyclic hydrocarbon group, more preferably an alkyl group or a polycyclic alicyclic saturated hydrocarbon group, and still more preferably a methyl group or a norbornyl group.

R^{Z} represents preferably a chain hydrocarbon group, more preferably an alkyl group, and still more preferably a methyl group.

Further, it is also preferred that R^{Y} and R^{Z} taken together represent the saturated aliphatic ring having 3 to 20 ring atoms together with the carbon atom to which R^{Y} and R^{Z} bond. The saturated aliphatic ring is preferably a monocyclic saturated aliphatic ring, and more preferably a cyclopentane ring or a cyclohexane ring.

The structural unit (I) is preferably a structural unit represented by one of the following formulae (2-1-1) to (2-1-3) and (2-2-1).

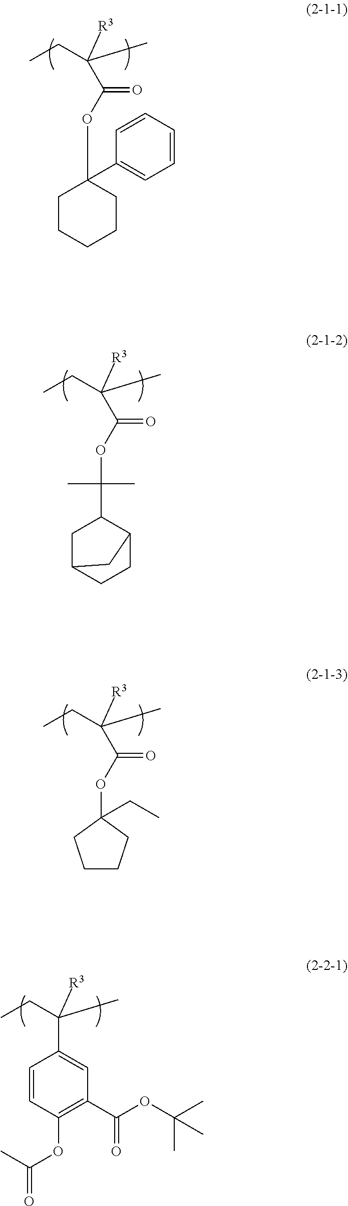

In the above formulae (2-1-1) to (2-1-3) and (2-2-1), R³ is as defined in the above formulae (2-1) to (2-2).

The lower limit of a proportion of the structural unit (I) contained in the polymer (A) is, with respect to total structural units constituting the polymer (A), preferably 20 mol % more preferably 25 mol % still more preferably 30 mol % and even further preferably 35 mol %. The upper limit of the proportion is preferably 90 mol %, more preferably 80 mol %, and still more preferably 75 mol %. When the proportion of the structural unit (I) falls within the above range, sensitivity to exposure light, CDU performance, and resolution resulting from of the radiation-sensitive resin composition can be further improved. It is to be noted that with respect to descriptions of the upper limit and the lower limit of numerical ranges as referred to herein, unless otherwise specified particularly, the upper limit may have the meaning of either “no greater than” or “less than”, and the lower limit may have the meaning of either “no less than” or “greater than”. Further, the upper limit value and the lower limit value may be combined ad libitum.

It is to be noted that there may be a case in which the structural unit contained in the polymer (A) is considered to fall under two or more categories of the structural unit, being overlapped. For example, a structural unit which is considered to fall under not only a category of structural unit (I), but also a category of one of the structural units other than the structural unit (I) can be contained. Referring to a specific example, a structural unit represented by the following formula falls under not only the category of the structural unit (I) as a “structural unit having an acid-labile group”, but also a category of a structural unit (III) as a “structural unit having an alcoholic hydroxy group” described later. With respect to such a structural unit, as referred to herein, the structural unit is defined to fall under the category of the structural unit denoted by the smaller number in parentheses. More specifically, the structural unit represented by the following formula is defined to fall under the category of the structural unit (I) as the “structural unit having an acid-labile group” although it is a structural unit having an alcoholic hydroxy group.

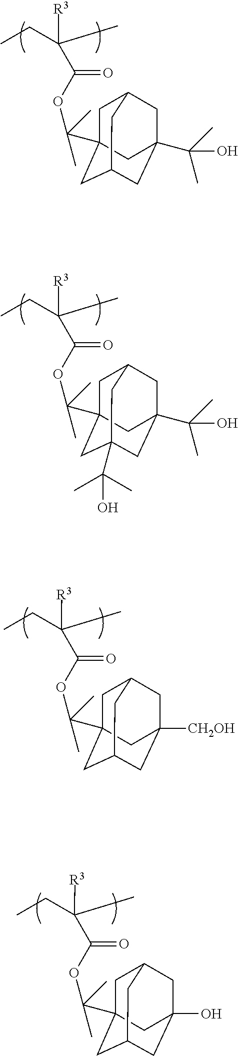

In the above formulae, R³ is as defined in the above formulae (2-1) to (2-2).

Structural Unit (II)

The structural unit (II) is a structural unit that includes a phenolic hydroxy group. The “phenolic hydroxy group” as referred to herein means a hydroxy group directly bonding to an aromatic ring in general, without being limited to a hydroxy group directly bonding to a benzene ring. The structural unit (II) is a structural unit that differs from the structural unit (I). A structural unit that includes both the phenolic hydroxy group and the acid-labile group is categorized to fall under the structural unit (I). In other words, the structural unit (II) does not include the acid-labile group. The polymer (A) may have one type, or two or more types of the structural unit (II).

In a case of conducting a KrF exposure, an EUV exposure, or an electron beam exposure, the sensitivity of the radiation-sensitive resin composition to exposure light can be further enhanced due to the polymer (A) having the structural unit (II). Therefore, in the case in which the polymer (A) has the structural unit (II), the radiation-sensitive resin composition can be suitably used as a radiation-sensitive resin composition for the KrF exposure, the EUV exposure or the electron beam exposure.

Examples of the structural unit (II) include structural units (hereinafter, may be also referred to as “structural units (II-1) to (II-17)”) represented by the following formulae (II-1) to (II-17), and the like.

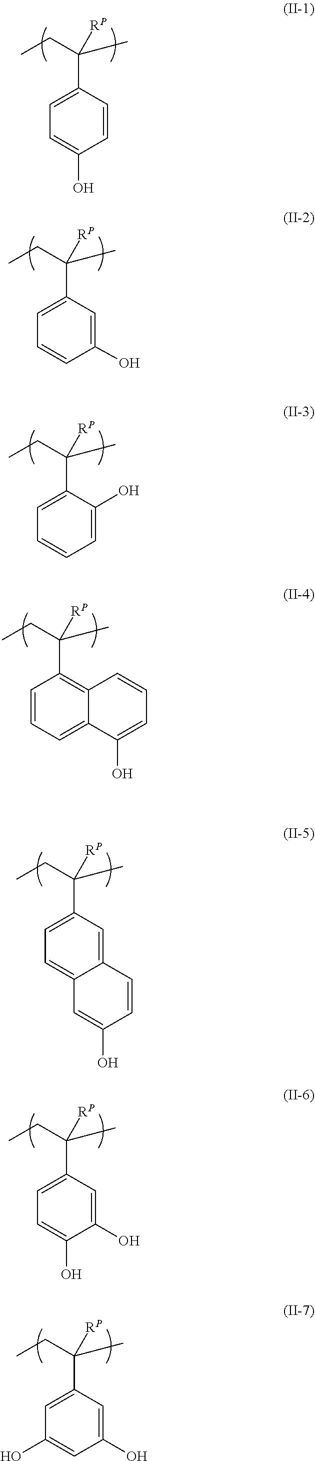

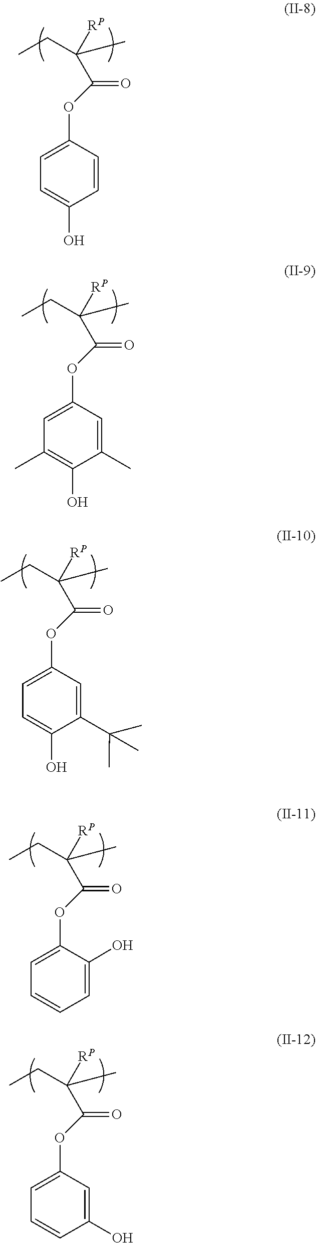

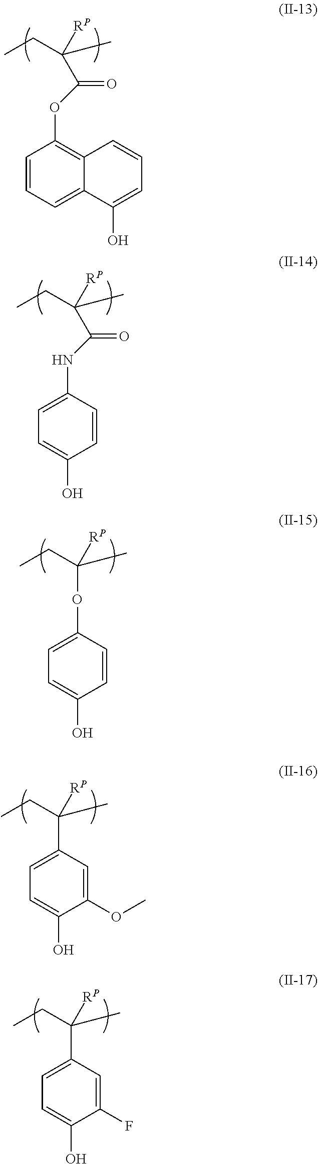

In the above formulae (II-1) to (II-17), R^{P} represents a hydrogen atom, a fluorine atom, a methyl group, or a trifluoromethyl group.

R^{P} represents preferably a hydrogen atom or a methyl group, in light of a degree of copolymerization of a monomer that gives the structural unit (II).

The structural unit (II) is preferably one of the structural units (II-1) to (II-3), (II-6) to (II-8), (II-11), and (II-12), or a combination of the same.

In the case in which the polymer (A) has the structural unit (II), the lower limit of a proportion of the structural unit (II) contained in the polymer (A) is, with respect to the total structural units constituting the polymer (A), preferably 15 mol %, more preferably 20 mol %, and still more preferably 25 mol %. The upper limit of the proportion is preferably 60 mol %, more preferably 50 mol %, still more preferably 40 mol %, and particularly preferably 35 mol %.

Other Structural Unit(s)

The other structural unit(s) is/are exemplified by a structural unit (hereinafter, may be also referred to as “structural unit (III)”) that includes an alcoholic hydroxy group, a structural unit (hereinafter, may be also referred to as “structural unit (IV)”) that includes an alkoxyalkyl group, a structural unit (hereinafter, may be also referred to as “structural unit (V)”) that includes a lactone structure, a cyclic carbonate structure, a sultone structure, or a combination of the same, and the like.

Structural Unit (III)

The structural unit (III) is a structural unit that includes an alcoholic hydroxy group. When the structural unit (III) is further incorporated, the solubility in a developer solution can be even further adequately adjusted.

Examples of the structural unit (III) include structural units represented by the following formulae, and the like.

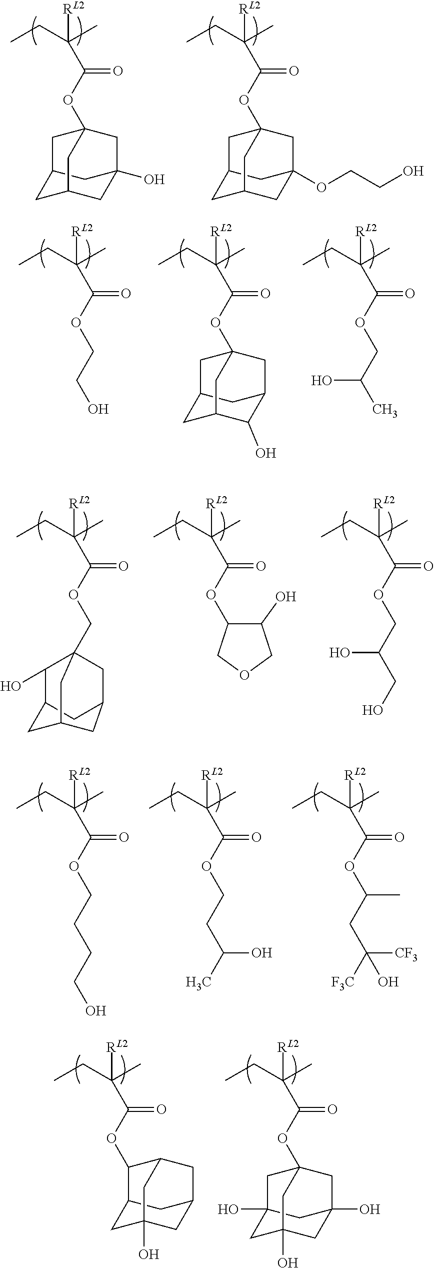

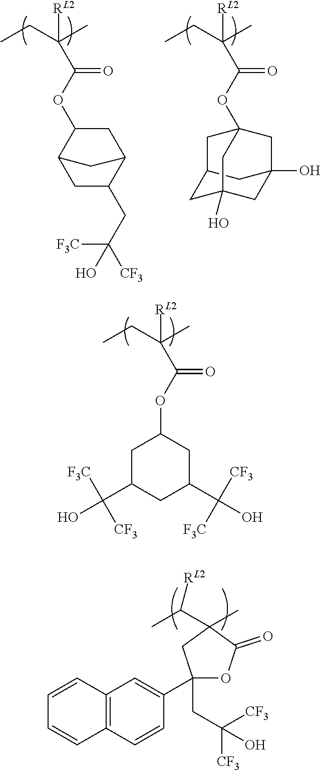

In the above formulae, R^{L2} represents a hydrogen atom, a fluorine atom, a methyl group, or a trifluoromethyl group.

Structural Unit (IV)

The structural unit (IV) is a structural unit that includes an alkoxyalkyl group. When the structural unit (IV) is further incorporated, adhesiveness to a substrate can be improved.

The alkoxyalkyl group is exemplified by groups obtained by combining an alkoxy group having 1 to 5 carbon atoms with an alkanediyl group having 2 to 5 carbon atoms such as a methoxyethyl group or an ethoxyethyl group, and the like. In other words, the alkoxyalkyl group is exemplified by a group represented by —R⁴¹—O—R⁴² (wherein R⁴¹ represents an alkanediyl group having 2 to 5 carbon atoms, and R⁴² represents an alkyl group having 1 to 5 carbon atoms), and the like.

Examples of the structural unit (IV) include structural unit represented by the following formula, and the like.

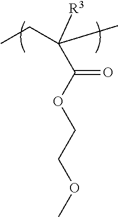

In the above formula, R^{L3} represents a hydrogen atom, a fluorine atom, a methyl group, or a trifluoromethyl group.

In the case in which the polymer (A) has the other structural unit(s), the lower limit of the proportion of the other structural unit(s) contained, with respect to the total structural units in the polymer (A) is, preferably 1 mol %, and more preferably 5 mol %. The upper limit of the proportion is preferably 30 mol %, and more preferably 20 mol %.

(B) Polymer

The polymer (B) is a polymer that differs from the polymer (A), and has a percentage content of fluorine atoms of greater than that of the polymer (A). In general, a more hydrophobic polymer than a polymer that serves as a base polymer tends to be localized in a resist film surface layer. Since the polymer (B) has a percentage content of fluorine atoms greater than that of the polymer (A), due to characteristics resulting from the hydrophobicity, the polymer (B) tends to be localized in the resist film surface layer. As a result, in the case in which the radiation-sensitive resin composition contains the polymer (B), a cross-sectional shape of a resist pattern to be formed is expected to be favorable. The radiation-sensitive resin composition may contain the polymer (B) as, for example, a surface conditioning agent of a resist film. The radiation-sensitive resin composition may contain one type, or two or more types of the polymer (B).

(C) Acid Generator

The acid generator (C) is a substance that generates an acid upon an exposure. The exposure light is exemplified by exposure light similar to those exemplified as the exposure light in the exposing step of the method of forming a resist pattern of an other embodiment of the present invention as described later, and the like. Due to the acid generated upon the exposure, the acid-labile group in the structural unit (I) contained in the polymer (A) is dissociated, whereby a difference is generated in the solubility in the developer solution, between light-exposed regions and light-unexposed regions of the resist film, and thus forming a resist pattern is enabled.

Examples of the acid generated from the acid generator (C) include sulfonic acid, imidic acid, and the like.

Regarding a form of the acid generator (C) contained in the radiation-sensitive resin composition, the form may be, for example: a low-molecular weight compound as described later (hereinafter, may be also referred to as “(C) acid generating agent” or “acid generating agent (C)”); a radiation-sensitive acid-generating polymer (hereinafter, may be also referred to as “(C) acid-generating polymer” or “acid-generating polymer (C)”); or both of these forms. The “low-molecular weight compound” as referred to herein means a compound having a molecular weight of no greater than 1,000, without being accompanied by molecular weight distribution. The “radiation-sensitive acid-generating polymer” as referred to herein means a polymer having a structural unit that generates an acid upon an exposure. In other words, the acid-generating polymer (C) may be also referred to as a form of the acid generator (C) incorporated as a part of the polymer. The acid-generating polymer (C) may be a polymer that differs from the polymer (A) and polymer (B). The radiation-sensitive resin composition may contain one type, or two or more types of the acid generator (C).

As the acid generator (C), an acid generating agent (C) is preferred. Examples of the acid generating agent (C) is exemplified by an onium salt compound, an N-sulfonyloxyimide compound, a sulfonimide compound, a halogen-containing compound, a diazo ketone compound, and the like.

Examples of the onium salt compound include a sulfonium salt, a tetrahydrothiophenium salt, an iodonium salt, a phosphonium salt, a diazonium salt, a pyridinium salt, and the like.

Specific examples of the acid generating agent (C) include compounds described in, for example, paragraphs [0080] to [0113] of Japanese Unexamined Patent Application, Publication No. 2009-134088, and the like.

The acid generating agent (C) is preferably an acid generating agent (C) that generates sulfonic acid upon an exposure. Examples of the acid generating agent (C) that generates sulfonic acid upon an exposure include a compound represented by the following formula (4), and the like.

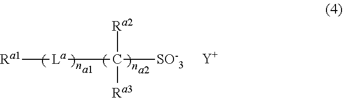

In the above formula (4): R^{a1} represents a monovalent organic group having 1 to 30 carbon atoms; L^{a} represents a divalent linking group; nₐ₁ is an integer of 0 to 10, wherein in a case in which nₐ₁ is no less than 2, a plurality of L^{a}s are identical or different from each other; R^{a2} and R^{a3} each independently represent a hydrogen atom, a fluorine atom, a monovalent hydrocarbon group having 1 to 20 carbon atoms, or a monovalent fluorinated hydrocarbon group having 1 to 20 carbon atoms; nₐ₂ is an integer of 0 to 10, wherein in a case in which nₐ₂ is no less than 2, a plurality of R^{a2}s are identical or different from each other, and a plurality of R^{a3}s are identical or different from each other; and Y⁺ represents a monovalent radiation-sensitive onium cation.

Examples of the monovalent organic group having 1 to 30 carbon atoms represented by R^{a1} in the above formula (2-2) include groups similar to the monovalent organic group exemplified as the monovalent organic group having 1 to 20 carbon atoms which may be represented by R⁴, and the like.

The monovalent organic group having 1 to 30 carbon atoms represented by Rai is preferably a monovalent group having 1 to 30 carbon atoms having a ring structure having 5 or more ring atoms. The ring structure having 5 or more ring atoms is exemplified by an aliphatic ring having 5 or more ring atoms, an aliphatic heteroring having 5 or more ring atoms, an aromatic hydrocarbon ring having 5 or more ring atoms, an aromatic heteroring having 5 or more ring atoms, and a combination of the same.

A part or all of hydrogen atoms contained in the ring structure may be substituted with a substituent. Examples of the substituent include halogen atoms such as a fluorine atom and an iodine atom, a hydroxy group, a carboxy group, a cyano group, a nitro group, an alkyl group, an alkoxy group, an alkoxycarbonyl group, an alkoxycarbonyloxy group, an acyl group, an acyloxy group, an oxo group (═O), and the like.

Examples of the aliphatic ring having 5 or more ring atoms include: monocyclic saturated aliphatic rings such as a cyclopentane ring, a cyclohexane ring, a cycloheptane ring, a cyclooctane ring, a cyclononane ring, a cyclodecane ring, and a cyclododecane ring; monocyclic unsaturated aliphatic rings such as a cyclopentene ring, a cyclohexene ring, a cycloheptene ring, a cyclooctene ring, and a cyclodecene ring; polycyclic saturated aliphatic rings such as a norbornane ring, an adamantane ring, a tricyclodecane ring, and a tetracyclododecane ring; polycyclic unsaturated aliphatic rings such as a norbornene ring and a tricyclodecene; and the like.

Examples of the aliphatic heteroring having 5 or more ring atoms include: lactone structures such as a hexanolactone ring and a norbornanelactone ring; sultone structures such as a hexanosultone ring and a norbornanesultone ring; oxygen atom-containing heterorings such as an oxacycloheptane ring and an oxanorbornane ring; nitrogen atom-containing heterorings such as an azacyclohexane ring and a diazabicyclooctane ring; sulfur atom-containing heterorings such as a thiacyclohexane ring and a thianorbornane ring; and the like.

Examples of the aromatic hydrocarbon ring having 5 or more ring atoms include a benzene ring, a naphthalene ring, a phenanthrene ring, an anthracene ring, and the like.

Examples of the aromatic heteroring having 5 or more ring atoms include: oxygen atom-containing heterorings such as a furan ring, a pyran ring, a benzofuran ring, and a benzopyran ring; nitrogen atom-containing heterorings such as a pyridine ring, a pyrimidine ring, and an indole ring; sulfur atom-containing heterorings such as a thiophene ring; and the like.

The lower limit of the number of ring atoms of the ring structure is preferably 6, more preferably 8, still more preferably 9, and particularly preferably 10. The upper limit of the number of ring atoms is preferably 25.

Examples of the divalent linking group represented by L^{a} include a carbonyl group, an ether group, a carbonyloxy group (—COO—), an oxycarbonyl group (—OCO—), a sulfide group, a thiocarbonyl group, a sulfonyl group, or a group obtained by combining the same, and the like. Of these, an ether group, a carbonyloxy group, or an oxycarbonyl group is preferred.

nₐ₁ is preferably 0 or 1.

Examples of the monovalent hydrocarbon group having 1 to 20 carbon atoms which may be represented by R^{a2} or R^{a3} include, among the monovalent organic groups having 1 to 20 carbon atoms which may be represented by R⁴ in the above formula (2-2), groups similar to the groups exemplified as the monovalent hydrocarbon group having 1 to 20 carbon atoms, and the like.

Examples of the monovalent fluorinated hydrocarbon group having 1 to 20 carbon atoms which may be represented by R^{a2} or R^{a3} include groups obtained from the monovalent hydrocarbon group having 1 to 20 carbon atoms by substituting a part or all of hydrogen atoms thereof with a fluorine atom, and the like.

R^{a2} and R^{a3} each represent preferably a hydrogen atom, a fluorine atom, the alkyl group, or the fluorinated alkyl group, and more preferably a hydrogen atom, a fluorine atom, a methyl group, or a trifluoromethyl group.

nₐ₂ is preferably 0 to 5, and still more preferably 0 to 4.

Examples of the monovalent radiation-sensitive onium cation represented by Y⁺ include monovalent cations (hereinafter, may be also referred to as “cations (r-a) to (r-c)”) represented by the following formulae (r-a) to (r-c), and the like.

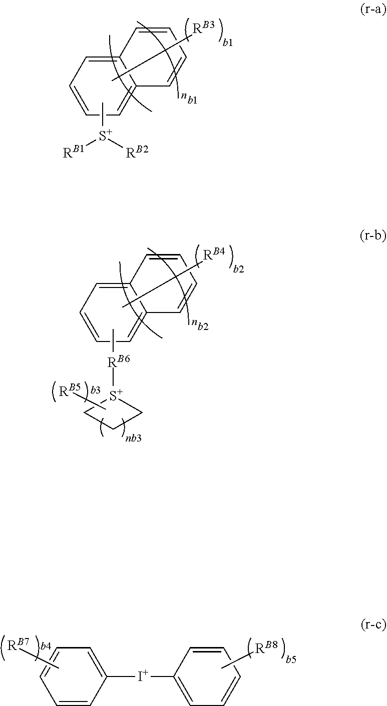

In the above formula (r-a), R^{B1} and R^{B2} each independently represent a group obtained by removing one hydrogen atom from a substituted or unsubstituted aromatic hydrocarbon ring having 6 to 20 ring atoms, or R^{B1} and R^{B2} taken together represent a substituted or unsubstituted polycyclic aromatic ring having 9 to 30 ring atoms together with the sulfur atom to which R^{B1} and R^{B2} bond; R^{B3} represents a halogen atom, a hydroxy group, a nitro group, or a monovalent organic group having 1 to 20 carbon atoms; b1 is an integer of 0 to 9, wherein in a case in which b1 is no less than 2, a plurality of R^{B3}s are identical or different from each other; and n_{b1} is an integer of 0 to 3.

In the above formula (r-b), R^{B4} and R^{B5} each independently represent a halogen atom, a hydroxy group, a nitro group, or a monovalent organic group having 1 to 20 carbon atoms; b2 is an integer of 0 to 9, wherein in a case in which b2 is no less than 2, a plurality of R^{B4}s are identical or different from each other; b3 is an integer of 0 to 10, wherein in a case in which b3 is no less than 2, a plurality of R^{B5}s represent a halogen atom, a hydroxy group, a nitro group, or a monovalent organic group having 1 to 20 carbon atoms, and are identical or different from each other; R^{B6} represents a single bond or a divalent organic group having 1 to 20 carbon atoms; n_{b2} is an integer of 0 to 2; and n_{b3} is an integer of 0 to 3.

In the above formula (r-c), R^{B7} and R^{B8} each independently represent a halogen atom, a hydroxy group, a nitro group, or a monovalent organic group having 1 to 20 carbon atoms; b4 is an integer of 0 to 5, wherein in a case in which b4 is no less than 2, a plurality of R^{B7}s are identical or different from each other; and b5 is an integer of 0 to 5, wherein in a case in which b5 is no less than 2, a plurality of R^{B8}s are identical or different from each other.

Examples of the aromatic hydrocarbon ring having 6 to 20 ring atoms that gives each of R^{B}1 and R^{B2} include rings similar to the ring exemplified as the aromatic hydrocarbon ring having 6 to 20 ring atoms that gives Ar² in the above formula (2-2), and the like. The aromatic hydrocarbon ring having 6 to 20 ring atoms that gives each of R^{B1} and R^{B2} is preferably a benzene ring or a naphthalene ring, and more preferably a benzene ring.

The polycyclic aromatic ring having 9 to 30 ring atoms which may be represented by R^{B}1 and R^{B2}, taken together, together with the sulfur atom to which R^{B}1 and R^{B2} bond is preferably a benzothiophene ring, a dibenzothiophene ring, a thioxanthene ring, a thiaxanthon ring, or a phenoxathiin ring, or the like.

In the aromatic hydrocarbon ring and the polycyclic aromatic ring, a part or all of hydrogen atoms bonding to atom(s) constituting these ring structures may be substituted with a substituent. Examples of the substituent include halogen atoms such as a fluorine atom and an iodine atom, a hydroxy group, a carboxy group, a cyano group, a nitro group, an alkyl group, a fluorinated alkyl group, an alkoxy group, an alkoxycarbonyl group, an alkoxycarbonyloxy group, an acyl group, an acyloxy group, an oxo group (═O) or a combination of the same, and the like. Of these, a fluorine atom, a hydroxy group, a trifluoromethyl group, a cyano group, a methyl group, or a tert-butyl group is preferred.

Examples of the monovalent organic group having 1 to 20 carbon atoms which may be represented by R^{B3}, R^{B4}, R^{B5}, R^{B7}, or R^{B8} include groups similar to the groups exemplified as the monovalent organic group having 1 to 20 carbon atoms which may be represented by R⁴ in the above formula (2-2), and the like.

Examples of the divalent organic group which may be represented by R^{B6} include groups obtained by removing one hydrogen atom from the group exemplified as the monovalent organic group having 1 to 20 carbon atoms which may be represented by R⁴ in the above formula (2-2), and the like.

b1 is preferably 0 to 2, more preferably 0 or 1, and still more preferably 0. n_{b1} is preferably 0 or 1.

The monovalent radiation-sensitive onium cation represented by Y⁺ is preferably the cation (r-a).

As the cation (r-a), cations represented by the following formulae (r-a-1) to (r-a-13) are preferred.

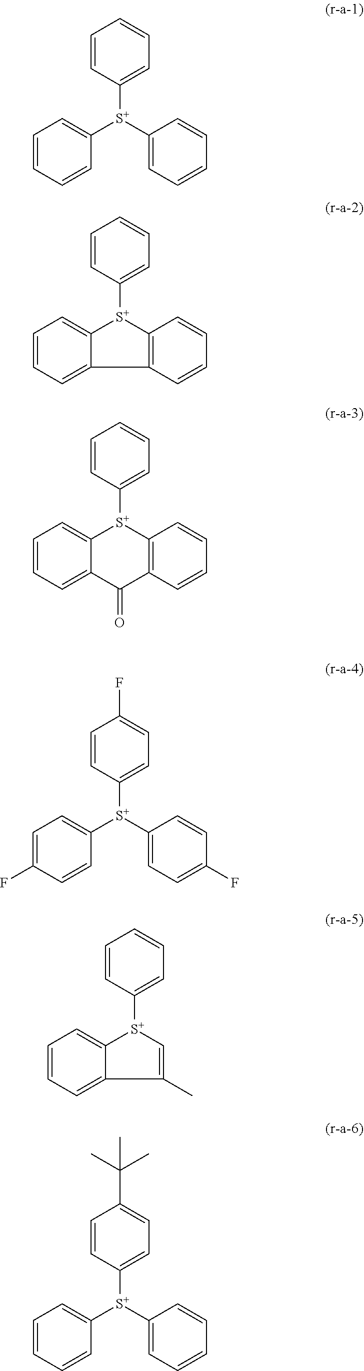 [SMILES: c1ccc([S+](c2ccccc2)c2ccccc2)cc1 ; c1ccc(-[s+]2c3ccccc3c3ccccc32)cc1 ; O=c1c2ccccc2[s+](-c2ccccc2)c2ccccc12 ; Fc1ccc([S+](c2ccc(F)cc2)c2ccc(F)cc2)cc1 ; Cc1c[s+](-c2ccccc2)c2ccccc12 ; CC(C)(C)c1ccc([S+](c2ccccc2)c2ccccc2)cc1]

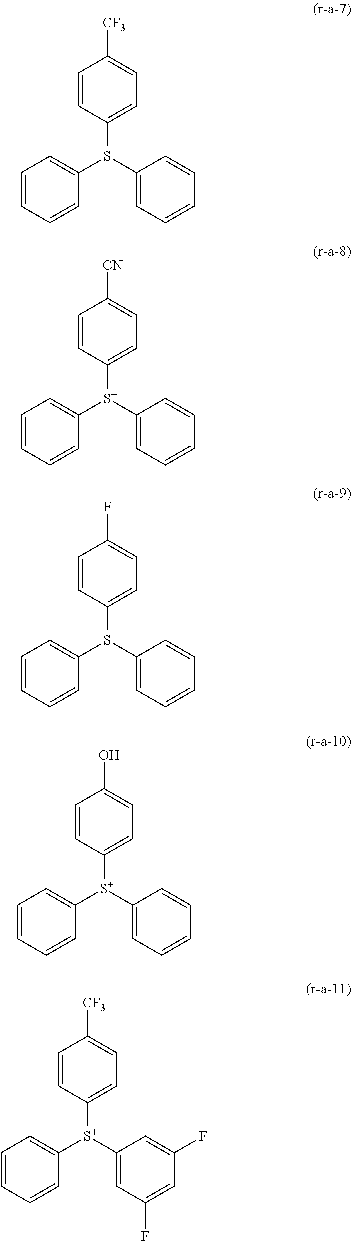 [SMILES: FC(F)(F)c1ccc([S+](c2ccccc2)c2ccccc2)cc1 ; N#Cc1ccc([S+](c2ccccc2)c2ccccc2)cc1 ; Fc1ccc([S+](c2ccccc2)c2ccccc2)cc1 ; Oc1ccc([S+](c2ccccc2)c2ccccc2)cc1 ; Fc1cc(F)cc([S+](c2ccccc2)c2ccc(C(F)(F)F)cc2)c1]

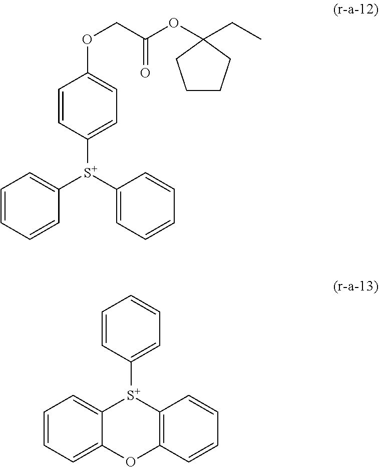 [SMILES: CCC1(OC(=O)COc2ccc([S+](c3ccccc3)c3ccccc3)cc2)CCCC1 ; c1ccc([S+]2c3ccccc3Oc3ccccc32)cc1]

As the acid generating agent (C), compounds represented by the following formulae (4-1) to (4-10) are preferred.

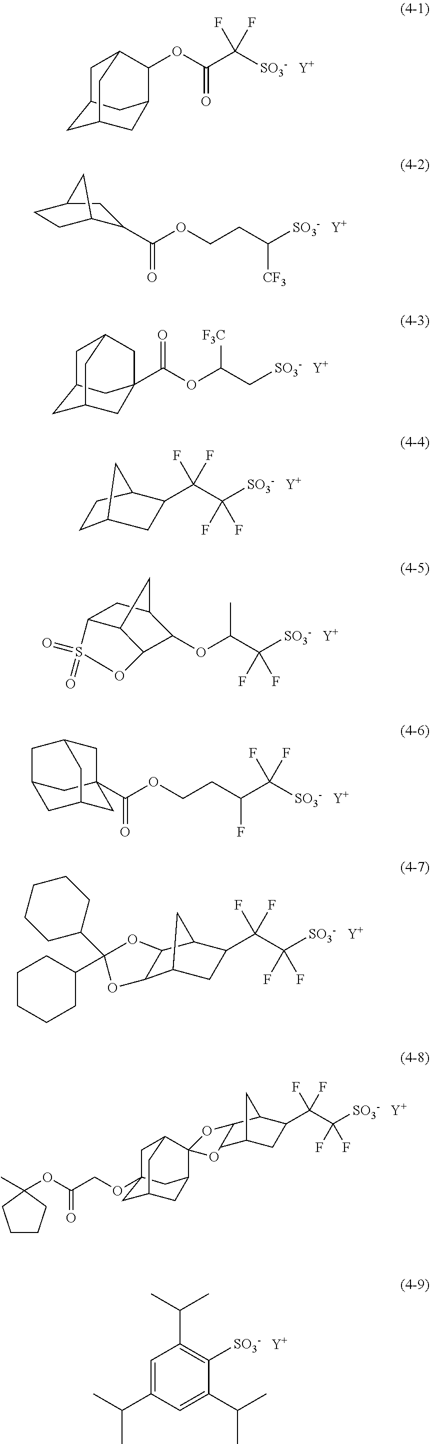

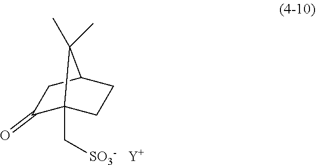

In the above formulae (4-1) to (4-10), Y⁺ is as defined in the above formula (4).

The lower limit of the content of the acid generating agent (C) in the radiation-sensitive resin composition is, with respect to 100 parts by mass of the polymer (A), preferably 5 parts by mass, more preferably 10 parts by mass, and still more preferably 15 parts by mass. The upper limit of the content is preferably 50 parts by mass, more preferably 40 parts by mass, and still more preferably 30 parts by mass. When the content of the acid generating agent (C) falls within the above range, the sensitivity of the radiation-sensitive resin composition to exposure light, CDU performance and resolution can be further improved.

(D) Compound

The compound (D) is a compound represented by the following formula (1). The compound (D) serves as an acid diffusion control agent (quencher). The acid diffusion control agent controls a diffusion phenomenon in a resist film, of the acid generated from the acid generator (C) generated upon the exposure, whereby an undesirable chemical reaction (for example, a dissociation reaction of the acid-labile group) in light-unexposed regions is controlled. Due to containing the compound (D), the radiation-sensitive resin composition enables formation of a resist pattern with favorable sensitivity to exposure light and superiority in terms of the CDU performance and the resolution.

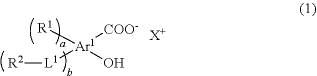

In the above formula (1), Ar¹ represents a group obtained by removing (a+b+2) hydrogen atoms from an aromatic hydrocarbon ring having 6 to 30 ring atoms; R¹ represents a halogen atom or a monovalent organic group having 1 to 20 carbon atoms; L¹ represents a divalent linking group; R² represents a substituted or unsubstituted monovalent aromatic hydrocarbon group having 6 to 20 carbon atoms; a is an integer of 0 to 10; b is an integer of 1 to 10, wherein a sum of a and b is no greater than 10, and wherein in a case in which a is no less than 2, a plurality of R¹s are identical or different from each other, and wherein in a case in which b is no less than 2, a plurality of Lis are identical or different from each other, and a plurality of R²s are identical or different from each other; and X⁺ represents a monovalent radiation-sensitive onium cation.

Examples of the aromatic hydrocarbon ring having 6 to 30 ring atoms that gives Ar¹ include rings similar to the rings exemplified as the aromatic hydrocarbon ring having 6 to 20 ring atoms that gives Ar² in the above formula (2-2), and the like. The aromatic hydrocarbon ring having 6 to 30 ring atoms that gives Ar¹ is preferably a benzene ring or a naphthalene ring.

The carboxylate group (—COO—) and the hydroxy group in the above formula (1) preferably bond to adjacent carbon atoms, respectively, constituting Ar. In other words, the carboxylate group and the hydroxy group preferably bond to each of ortho positions of an identical benzene ring in Ar¹. To further paraphrase, it is preferred that the carbon atom on Ar¹ to which the carboxylate group bonds is directly linked to the carbon atom on Ar¹ to which the hydroxy group bonds. In this case, storage stability of the radiation-sensitive resin composition can be improved.

Examples of the monovalent organic group having 1 to 20 carbon atoms which may be represented by R¹ include groups similar to the groups exemplified as the monovalent organic group having 1 to 20 carbon atoms which may be represented by R⁴ in the above formula (2-2), and the like.

The halogen atom which may be represented by R¹ is a fluorine atom, a chlorine atom, a bromine atom, or an iodine atom.

R¹ represents preferably the halogen atom, and more preferably an iodine atom.

a is preferably 0 to 2, more preferably 0 or 1, and still more preferably 0.

Examples of the divalent linking group represented by L¹ include an ether group (—O—), a carbonyloxy group (—CO—O—), an oxycarbonyl group (—O—CO—), a carbonyl sulfide group (—CO—S—), a sulfide group (—S—), a thiocarbonyl group (—CS—), a sulfonyl group (—SO₂—), an alkanediyl group having 1 to 5 carbon atoms, or a group being a combination thereof, and the like.

L¹ represents preferably a carbonyloxy group, an oxycarbonyl group, an ether group, a sulfide group, an alkanediyl group having 1 to 5 carbon atoms, or a group being a combination thereof, and more preferably a carbonyloxy group, a methanediyloxy group (—CH₂—O—), a methanediylsulfide group (—CH₂—S—), or an oxycarbonylmethanediyloxy group (—O—CO—CH₂—O—).

Furthermore, when the aforementioned divalent linking groups are to be combined, there is a case in which it is preferred to avoid combining the groups of a similar type. In other words, there is a case in which L¹ represents preferably a carbonyloxy group, an oxycarbonyl group, an ether group, a sulfide group, an alkanediyl group having 1 to 5 carbon atoms, or a group being a combination thereof (wherein any combination of two or more groups of the same type is excluded). It is to be noted that, for example, the oxycarbonylmethanediyloxy group is considered to be a group being a combination of two ether groups, a carbonyl group, and a methanediyl group. However, the divalent linking group exemplified above does not include a “carbonyl group”. Thus, the oxycarbonylmethanediyloxy group is regarded as a group being a combination of an oxycarbonyl group, a methanediyl group, and an ether group, thereby being defined not to fall under the case of a combination of two or more groups of the same type. On the other hand, for example, an oxymethanediyloxy group (—O—CH₂—O—) is a group being a combination of two ether groups, thereby being defined to fall under the case of a combination of two or more groups of the same type.

Examples of the monovalent aromatic hydrocarbon group having 6 to 20 carbon atoms in R² include, among the monovalent organic groups having 1 to 20 carbon atoms which may be represented by R⁴ in the above formula (2-2), groups similar to the groups exemplified as the monovalent aromatic hydrocarbon group having 6 to 20 carbon atoms, and the like.

R² represents preferably a substituted or unsubstituted aryl group, and more preferably a substituted or unsubstituted phenyl group, or a substituted or unsubstituted naphthyl group.

Examples of the substituent which may be incorporated in the aromatic hydrocarbon group represented by R² include halogen atoms such as a fluorine atom and an iodine atom, a hydroxy group, a carboxy group, a cyano group, a nitro group, an alkoxy group, an alkoxycarbonyl group, an alkoxycarbonyloxy group, an acyl group, an acyloxy group, and the like. Of these, a fluorine atom, an iodine atom, or an alkoxy group is preferred, and an iodine atom is more preferred.

R² represents further preferably a monovalent aromatic hydrocarbon group having 6 to 20 carbon atoms in which at least one hydrogen atom on an aromatic ring is substituted with an iodine atom. In this case, the sensitivity to exposure light can be further improved.

Examples of the monovalent radiation-sensitive onium cation represented by X⁺ include monovalent radiation-sensitive onium cations exemplified as Y⁺ in the above formula (4), and the like. X⁺ represents preferably the above cation (r-a), and more preferably any one of the cations (r-a-1) to (r-a-13).

As the compound (D), compounds represented by the following formulae (1-1) to (1-14) are preferred.

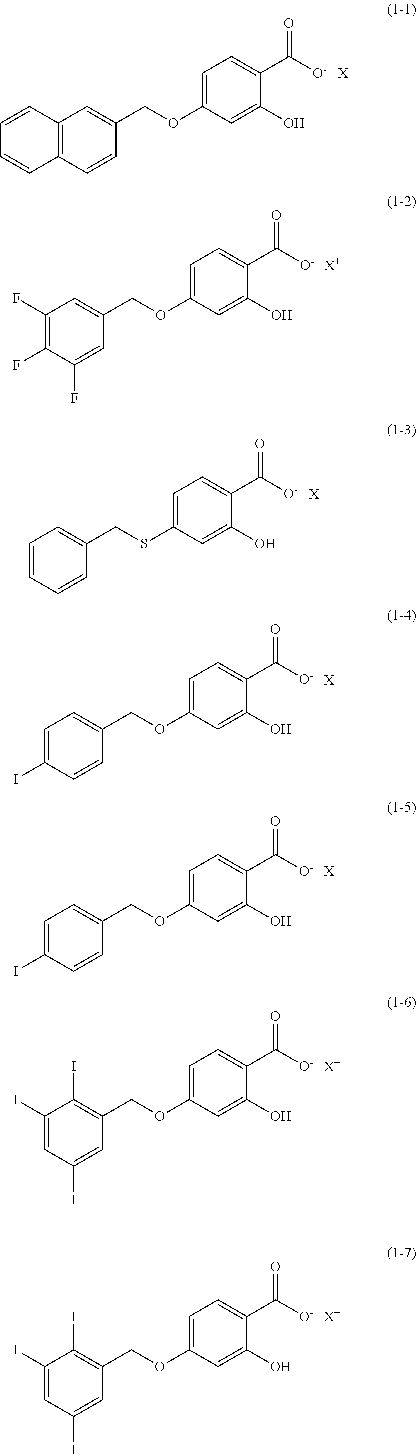

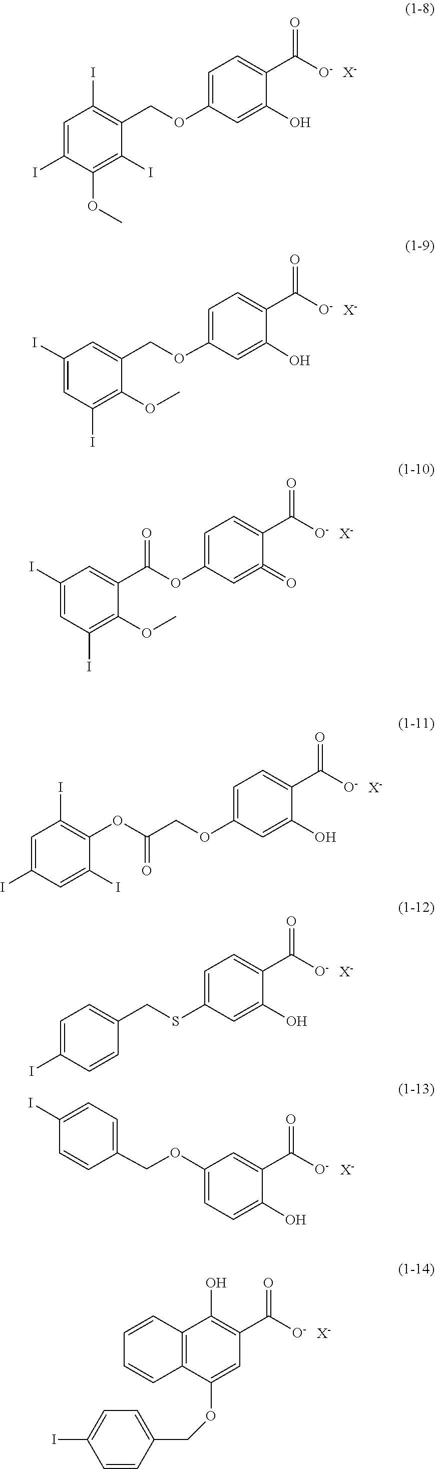

In the above formulae (1-1) to (1-14), X⁺ is as defined in the above formula (1).

The lower limit of the proportion of the compound (D) contained in the radiation-sensitive resin composition is, with respect to 100 mol % acid generator (C), preferably 1 mol %, more preferably 5 mol %, and still more preferably 10 mol %. The upper limit of the proportion is preferably 200 mol %, more preferably 100 mol %, still more preferably 50 mol %, and particularly preferably 25 mol %. When the proportion of the compound (D) falls within the above range, the sensitivity to exposure light, as well as the CDU performance and the resolution of the resist pattern formed from the radiation-sensitive resin composition can be further improved.

(E) Organic Solvent

The radiation-sensitive resin composition typically contains the organic solvent (E). The organic solvent (E) is not particularly limited as long as it is a solvent capable of dissolving or dispersing at least the polymer (A), the acid generator (C), and the compound (D), as well as the other optional component(s) which may be contained as needed.

The organic solvent (E) is exemplified by an alcohol solvent, an ether solvent, a ketone solvent, an amide solvent, an ester solvent, a hydrocarbon solvent, and the like. The radiation-sensitive resin composition may contain one, or two or more types of the organic solvent (E).

Examples of the alcohol solvent include:

aliphatic monohydric alcohol solvents having 1 to 18 carbon atoms such as 4-methyl-2-pentanol and n-hexanol;

alicyclic monohydric alcohol solvents having 3 to 18 carbon atoms such as cyclohexanol;

polyhydric alcohol solvents having 2 to 18 carbon atoms such as 1,2-propylene glycol;

polyhydric alcohol partial ether solvents having 3 to 19 carbon atoms such as propylene glycol monomethyl ether; and the like.

Examples of the ether solvent include:

dialkyl ether solvents such as diethyl ether, dipropyl ether, dibutyl ether, dipentyl ether, diisoamyl ether, dihexyl ether, and diheptyl ether;

cyclic ether solvents such as tetrahydrofuran and tetrahydropyran;

aromatic ring-containing ether solvents such as diphenyl ether and anisole; and the like.

Examples of the ketone solvent include:

chain ketone solvents such as acetone, methyl ethyl ketone, methyl n-propyl ketone, methyl n-butyl ketone, diethyl ketone, methyl iso-butyl ketone, 2-heptanone, ethyl n-butyl ketone, methyl n-hexyl ketone, di-iso-butyl ketone, and trimethylnonanone;

cyclic ketone solvents such as cyclopentanone, cyclohexanone, cycloheptanone, cyclooctanone, and methylcyclohexanone;

2,4-pentanedione, acetonylacetone, and acetophenone; and the like.

Examples of the amide solvent include:

cyclic amide solvents such as N,N′-dimethylimidazolidinone and N-methylpyrrolidone;

chain amide solvents such as N-methylformamide, N,N-dimethylformamide, N,N-diethylformamide, acetamide, N-methylacetamide, N,N-dimethylacetamide, and N-methylpropionamide; and the like.

Examples of the ester solvent include:

monocarboxylic acid ester solvents such as n-butyl acetate and ethyl lactate;

lactone solvents such as γ-butyrolactone and valerolactone;

polyhydric alcohol carboxylate solvents such as propylene glycol acetate;

polyhydric alcohol partial ether carboxylate solvents such as propylene glycol monomethyl ether acetate;

polyhydric carboxylic acid diester solvents such as diethyl oxalate;

carbonate solvents such as dimethyl carbonate and diethyl carbonate; and the like.

Examples of the hydrocarbon solvent include:

aliphatic hydrocarbon solvents having 5 to 12 carbon atoms such as n-pentane and n-hexane;

aromatic hydrocarbon solvents having 6 to 16 carbon atoms such as toluene and xylene; and the like.

The organic solvent (E) is preferably the alcohol solvent, the ester solvent, or a combination of the same, more preferably the polyhydric alcohol partial ether solvent having 3 to 19 carbon atoms, the polyhydric alcohol partial ether carboxylate solvent, or a combination of the same, and still more preferably propylene glycol 1-monomethyl ether, propylene glycol monomethyl ether acetate, or a combination of the same.

In the case of the radiation-sensitive resin composition containing the organic solvent (E), the lower limit of a proportion of the organic solvent (E) with respect to total components contained in the radiation-sensitive resin composition is preferably 50% by mass, more preferably 60% by mass, still more preferably 70% by mass, and particularly preferably 80% by mass. The upper limit of the proportion is preferably 99.9% by mass, more preferably 99.5% by mass, and still more preferably 99.0% by mass.

Other Optional Component(s)

The other optional component(s) is/are exemplified by an acid diffusion control agent other than the compound (D) described above, a surfactant, and the like. The radiation-sensitive resin composition may contain one, or two or more types each of the other optional component(s).

Method of Forming Resist Pattern

The method of forming a resist pattern according to the other embodiment of the present invention includes: a step (hereinafter, may be also referred to as “applying step”) of applying a radiation-sensitive resin composition directly or indirectly on a substrate; a step (hereinafter, may be also referred to as “exposing step”) of exposing a resist film formed by the applying step; and a step (hereinafter, may be also referred to as “developing step”) of developing the resist film exposed.

According to the method of forming a resist film, due to using the radiation-sensitive resin composition of the one embodiment of the present invention as the radiation-sensitive resin composition in the applying step, formation of a resist pattern with favorable sensitivity to exposure light and superiority in terms of the CDU performance and the resolution is enabled.

Each step included in the method of forming a resist pattern will be described below.

Applying Step

In this step, the radiation-sensitive resin composition is applied directly or indirectly on the substrate. By this step, the resist pattern is formed directly or indirectly on the substrate.

In this step, the radiation-sensitive resin composition of the one embodiment of the present invention, described above, is used as the radiation-sensitive resin composition.

The substrate is exemplified by a conventionally well-known substrate such as a silicon wafer, a wafer coated with silicon dioxide or aluminum, and the like.

An application procedure is exemplified by spin coating, cast coating, roll coating, and the like. After the application, prebaking (hereinafter, may be also referred to as “PB”) may be carried out as needed for evaporating the solvent remaining in the coating film. The lower limit of a PB temperature is preferably 60° C., and more preferably 80° C. The upper limit of the PB temperature is preferably 150° C., and more preferably 140° C. The lower limit of a PB time period is preferably 5 sec, and more preferably 10 sec. The upper limit of the PB time period is preferably 600 sec, and more preferably 300 sec. The lower limit of an average thickness of the resist film formed is preferably 10 nm, and more preferably 20 nm. The upper limit of the average thickness is preferably 1,000 nm, and more preferably 500 nm.

Exposing Step

In this step, the resist film formed by the applying step is exposed. This exposure is carried out by irradiation with an exposure light through a photomask (as the case may be, through a liquid immersion medium such as water). As the exposure light, far ultraviolet rays, EUV, or electron beams are preferred; an ArF excimer laser beam (wavelength: 193 nm), a KrF excimer laser beam (wavelength: 248 nm), EUV (wavelength: 13.5 mm), or an electron beam is more preferred; a KrF excimer laser beam, EUV, or an electron beam is still more preferred; and EUV or an electron beam is particularly preferred.

It is preferred that post exposure baking (hereinafter, may be also referred to as “PEB”) is carried out after the exposure. This PEB enables an increase in a difference in solubility of the resist film in a developer solution between the light-exposed regions and light-unexposed regions. The lower limit of a PEB temperature is preferably 50° C., more preferably 80° C., and still more preferably 100° C. The upper limit of the PEB temperature is preferably 180° C., and more preferably 130° C. The lower limit of a PEB time period is preferably 5 sec, more preferably 10 sec, and still more preferably 30 sec. The upper limit of the PEB time period is preferably 600 sec, more preferably 300 sec, and still more preferably 100 sec.

Developing Step

In this step, the resist film exposed is developed. Accordingly, formation of a predetermined resist pattern is enabled. The development procedure in the developing step may be carried out by either development with an alkali, or development with an organic solvent.

In the case of the development with an alkali, the developer solution for use in the development is exemplified by: alkaline aqueous solutions prepared by dissolving at least one alkaline compound such as sodium hydroxide, potassium hydroxide, sodium carbonate, sodium silicate, sodium metasilicate, aqueous ammonia, ethylamine, n-propylamine, diethylamine, di-n-propylamine, triethylamine, methyldiethylamine, ethyldimethylamine, triethanolamine, tetramethylammonium hydroxide (hereinafter, may be also referred to as “TMAH”), pyrrole, piperidine, choline, 1,8-diazabicyclo-[5.4.0]-7-undecene, and 1,5-diazabicyclo-[4.3.0]-5-nonene; and the like. Of these, an aqueous TMAH solution is preferred, and a 2.38% by mass aqueous TMAH solution is more preferred.

In the case of the development with an organic solvent, the developer solution is exemplified by: an organic solvent such as a hydrocarbon solvent, an ether solvent, an ester solvent, a ketone solvent, and an alcohol solvent; a solution containing the organic solvent; and the like. An exemplary organic solvent includes the solvents exemplified as the organic solvent (E) in the radiation-sensitive resin composition of the one embodiment of the present invention, and the like.

Compound

The compound of a still another embodiment of the present invention has been described as the compound (D) in the radiation-sensitive resin composition of the one embodiment of the present invention, described above. The compound may be suitably used as a component of a radiation-sensitive resin composition. In addition, the compound may be suitably used as an acid diffusion control agent.

### EXAMPLES

Hereinafter, the present invention is explained in detail by way of Examples, but the present invention is not in any way limited to these Examples. Measuring methods for various types of physical property values are shown below.

Weight Average Molecular Weight (Mw), Number Average Molecular Weight (Mn), and Dispersity Index (Mw/Mn)

Measurements of the Mw and the Mn of the polymer were carried out in accordance with the conditions described in the aforementioned paragraph “Method for Measuring Mw and Mn”. The dispersity index (Mw/Mn) of the polymer was calculated from the measurement results of the Mw and the Mn.

Synthesis of Compound (D)

### Synthesis Example 1-1: Synthesis of Compound (D-1)

In a vessel charged with N,N-dimethylformamide (10 mL), sodium hydride (41.6 mmol) was dispersed. To this vessel, a solution of 2,4-dihydroxybenzoic acid (10.0 mmol) in N,N-dimethylformamide (7.5 mL) was added dropwise at room temperature over 1 hour. Next, a solution of 2-(bromomethyl)naphthalene (10.0 mmol) in N,N-dimethylformamide (7.5 mL) was added dropwise at room temperature over 1 hour. After completion of the dropwise addition, the mixture was stirred at room temperature for an additional 2 hours. After cooling to 10° C. or below, the reaction was stopped by adding 1 mol/L hydrochloric acid (100 mL). A thus precipitated solid was filtered and washed with each of distilled water and methylene chloride to give a compound (2-hydroxy-4-(naphthalen-2-ylmethoxy)benzoate; hereinafter, may be also referred to as “compound (pD-1)”) represented by the following formula (pD-1).

The compound (pD-1) (8.20 mmol), sodium bicarbonate (16.4 mmol), triphenylsulfonium chloride (12.3 mmol), methylene chloride (82 mL), and distilled water (82 mmol) were admixed, and a resultant mixture was stirred at room temperature for 3 hours. After completion of the reaction, liquid separation was conducted and an organic layer was dried over anhydrous sodium sulfate, followed by filtration. The solvent was distilled away to give a compound (hereinafter, may be also referred to as “compound (D-1)”) represented by the following formula (D-1).

A synthesis scheme of the compound (D-1) is shown below.

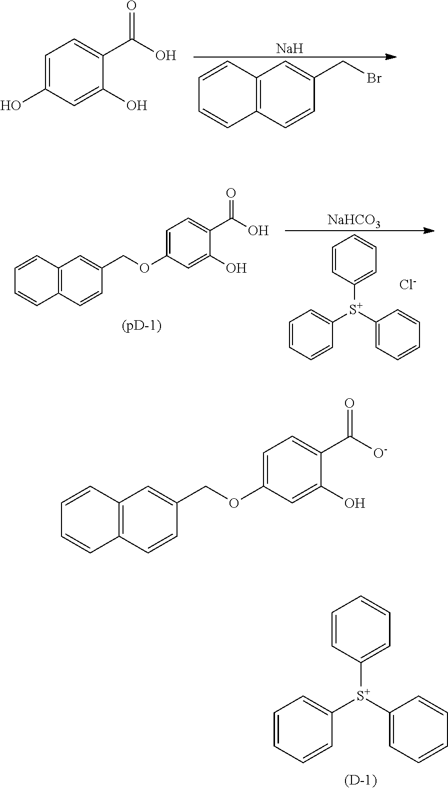

### Synthesis Examples 1-2 to 1-14: Syntheses of Compounds (D-2) to (D-14)

Similarly to Synthesis Example 1 except that the precursor was appropriately selected, compounds (hereinafter, may be also referred to as “compounds (D-2) to (D-14)”) represented by the following formulae (D-2) to (D-14) were synthesized.

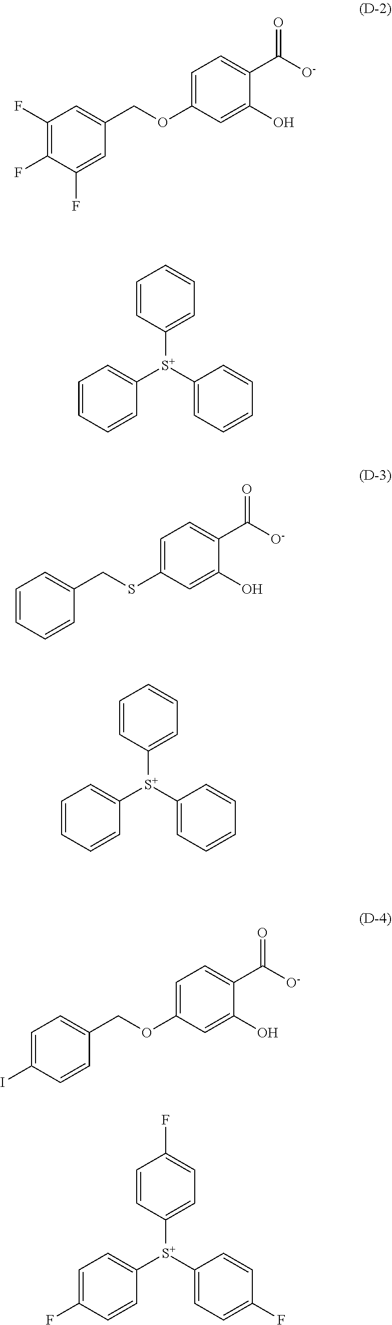 [SMILES: O=C([O-])c1ccc(OCc2cc(F)c(F)c(F)c2)cc1O ; c1ccc([S+](c2ccccc2)c2ccccc2)cc1 ; O=C([O-])c1ccc(SCc2ccccc2)cc1O ; c1ccc([S+](c2ccccc2)c2ccccc2)cc1 ; O=C([O-])c1ccc(OCc2ccc(I)cc2)cc1O ; Fc1ccc([S+](c2ccc(F)cc2)c2ccc(F)cc2)cc1]

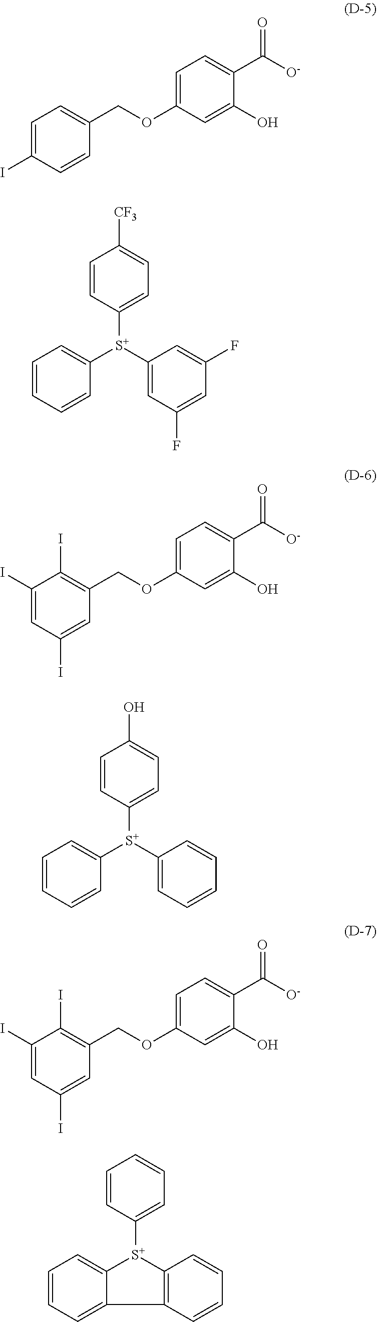 [SMILES: O=C([O-])c1ccc(OCc2ccc(I)cc2)cc1O ; Fc1cc(F)cc([S+](c2ccccc2)c2ccc(C(F)(F)F)cc2)c1 ; O=C([O-])c1ccc(OCc2cc(I)cc(I)c2I)cc1O ; Oc1ccc([S+](c2ccccc2)c2ccccc2)cc1 ; O=C([O-])c1ccc(OCc2cc(I)cc(I)c2I)cc1O ; c1ccc(-[s+]2c3ccccc3c3ccccc32)cc1]

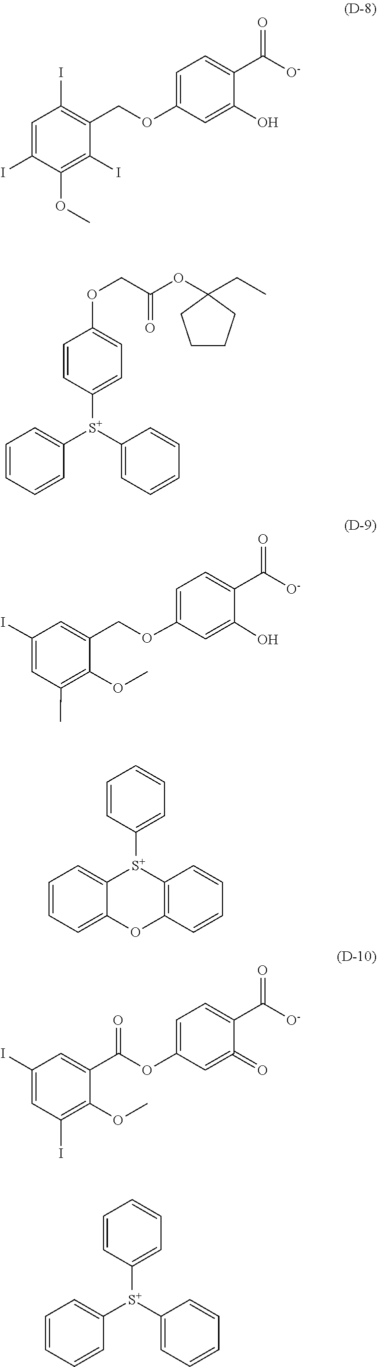

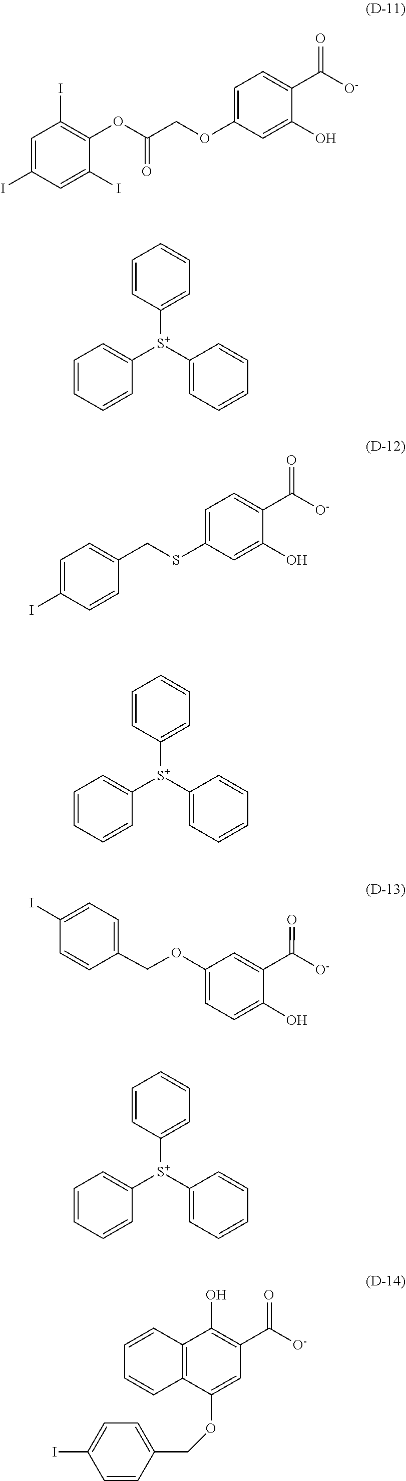

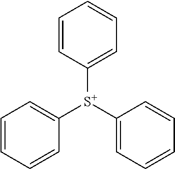 [SMILES: c1ccc([S+](c2ccccc2)c2ccccc2)cc1]

Synthesis of Polymer (A)

For syntheses of polymers (A), monomers (hereinafter, may be also referred to as “monomers (M-1) to (M-11)”) represented by the following formulae (M-1) to (M-11) were used. In the following Synthesis Examples, unless otherwise specified particularly, the term “parts by mass” means a value, provided that the total mass of the monomers used was 100 parts by mass, and the term “mol %” means a value, provided that the total mol number of the monomers used was 100 mol %.

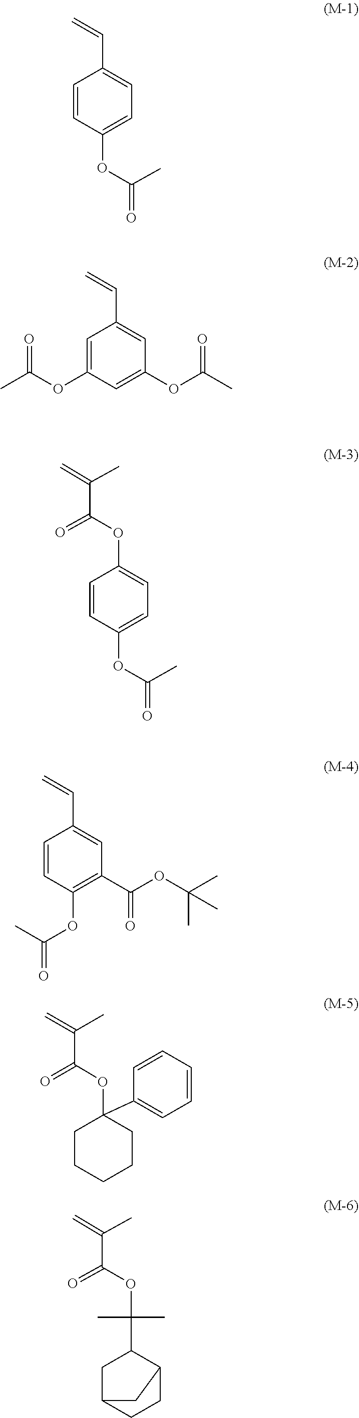

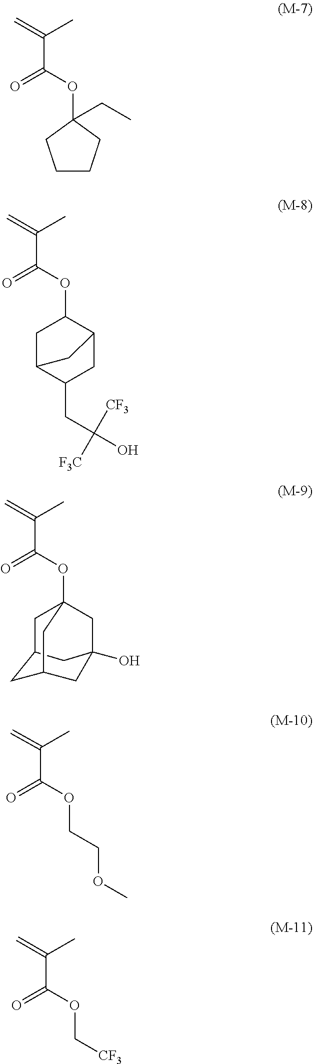

### Synthesis Example 2-1: Synthesis of Polymer (A-1)

The monomer (M-1), the monomer (M-5), and the monomer (M-8) were dissolved in propylene glycol-1-monomethyl ether (200 parts by mass) such that a molar ratio of the monomers became 45/45/10. Thereto was added as an initiator, 7 mol % 2,2′-azobis(methylisobutyrate) to prepare a monomer solution. Meanwhile, propylene glycol monomethyl ether (100 parts by mass with respect to a total amount of the monomers) was charged into an empty vessel, and the mixture was heated to 85° C. with stirring. Into this vessel, the monomer solution was added dropwise over 3 hours. After completion of the dropwise addition, the mixture was heated at 85° C. for an additional 3 hours, and then the polymerization solution was cooled to room temperature. The polymerization solution was added dropwise into n-hexane (1,000 parts by mass) to allow for solidification purification of the polymer.

The polymer was added again to propylene glycol monomethyl ether (150 parts by mass) and dissolved. Thereto were added methanol (150 parts by mass), triethylamine (1.5 molar equivalent with respect to the using amount of the compound (M-1)) and water (1.5 molar equivalent with respect to the using amount of the compound (M-1)). Reflux was allowed at a boiling point for 8 hours to carry out a hydrolysis reaction. After completion of the reaction, the solvent and triethylamine were distilled off under reduced pressure, and a thus obtained polymer was dissolved in acetone (150 parts by mass). This solution was added dropwise into water (2,000 parts by mass) to permit coagulation, and thus produced white powder was filtered. The white powder was dried at 50° C. for 17 hours to give a white powdery polymer (A-1) with a favorable yield. With respect to the polymer (A-1), the Mw was 7,200, and the Mw/Mn was 1.5.

### Synthesis Examples 2-2 to 2-8: Syntheses of Polymers (A-2) to (A-8)

Similarly to Synthesis Example 1 except that each monomer of the type and in a blend proportion as shown in Table 1 below was used, polymers (A-2) to (A-8) were synthesized.

TABLE 1

| | | Monomer that | | Monomer that | | Monomer that | | | |
|---|---|---|---|---|---|---|---|---|---|
| | | gives structural | | gives structural | | gives other | | | |
| | | unit (I) | | unit (II) | | structural unit(s) | | | |
| | | | using | | using | | using | | |
| | | | amount | | amount | | amount | | |
| | (A) | | (% by | | (% by | | (% by | | |
| | Polymer | type | mole) | type | mole) | type | mole) | Mw | Mw/Mn |
| Synthesis | A-1 | M-5 | 45 | M-1 | 45 | M-8 | 10 | 7,200 | 1.5 |
| Example 2-1 | | | | | | | | | |
| Synthesis | A-2 | M-5 | 55 | M-1 | 25 | — | — | 5,300 | 1.4 |
| Example 2-2 | | | | M-2 | 20 | | | | |
| Synthesis | A-3 | M-6 | 55 | M-2 | 45 | — | — | 4,200 | 1.3 |
| Example 2-3 | | | | | | | | | |
| Synthesis | A-4 | M-7 | 40 | M-2 | 30 | — | — | 6,800 | 1.6 |
| Example 2-4 | | | | M-3 | 30 | | | | |
| Synthesis | A-5 | M-7 | 70 | M-1 | 30 | — | — | 5,700 | 1.5 |
| Example 2-5 | | | | | | | | | |
| Synthesis | A-6 | M-6 | 50 | M-1 | 35 | M-9 | 15 | 8,500 | 1.5 |
| Example 2-6 | | | | | | | | | |
| Synthesis | A-7 | M-4 | 60 | M-1 | 30 | M-10 | 10 | 6,300 | 1.5 |
| Example 2-7 | | | | | | | | | |
| Synthesis | A-8 | M-4 | 30 | M-1 | 35 | — | — | 4,800 | 1.4 |
| Example 2-8 | | M-7 | 35 | | | | | | |

Synthesis of Polymer (B)

### Synthesis Example 3-1: Synthesis of Polymer (B-1)

The monomer (M-7) and the monomer (M-11) were dissolved in 2-butanone (100 parts by mass) such that a molar ratio of the monomers became 70/30. Thereto was added as an initiator, 5 mol % azobisisobutyronitrile to prepare a monomer solution. On the other hand, 2-butanone (50 parts by mass) was charged into an empty vessel, and nitrogen was purged for 30 min. The interior of the vessel was heated to 80° C., and the monomer solution was added dropwise over 3 hrs with stirring. After completion of the dropwise addition, the mixture was heated at 80° C. for an additional 3 hours, and then the polymerization solution was water cooled to 30° C. or below. After the polymerization solution was transferred into a separatory funnel, hexane (150 parts by mass) was added to uniformly dilute the polymerization solution. Furthermore, methanol (600 parts by mass) and water (30 parts by mass) were charged and mixed. After the mixture was left to stand for 30 min, the underlayer was recovered and the solvent was replaced with propylene glycol monomethyl ether acetate. Thus, a solution containing the polymer (B-1) in propylene glycol monomethyl ether acetate was obtained. With respect to the polymer (B-1), the Mw was 7,800, and the Mw/Mn was 1.8.

Preparation of Radiation-Sensitive Resin Composition

The acid generating agent (C), the acid diffusion control agent (D), and the organic solvent (E) used in preparation of the radiation-sensitive resin composition are shown below. In the following Examples and Comparative Examples, unless otherwise specified particularly, the term “parts by mass” means a value, provided that the mass of the polymer (A) used was 100 parts by mass, and the term “mol %” means a value, provided that the mol number of the acid generating agent (C) used was 100 mol %.

(C) Acid Generating Agent

Compounds (hereinafter, may be also referred to as “acid generating agents (C-1) to (C-10)”) represented by the following formulae (C-1) to (C-10) were used as the acid generating agent (C).

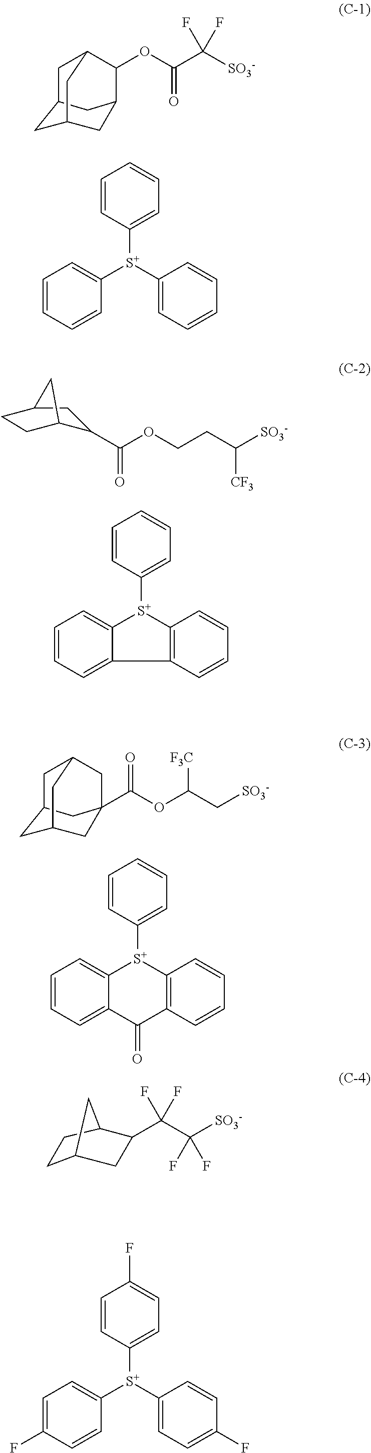

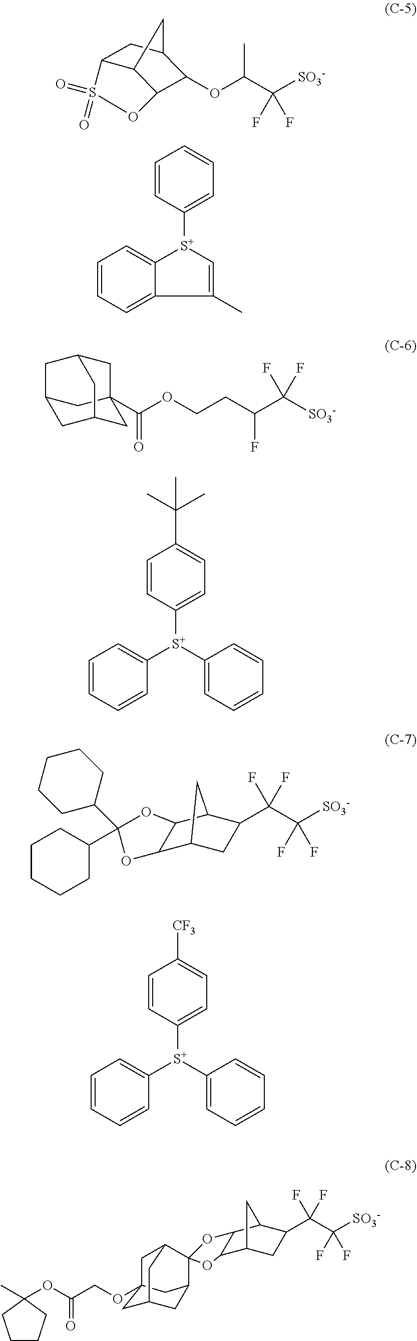

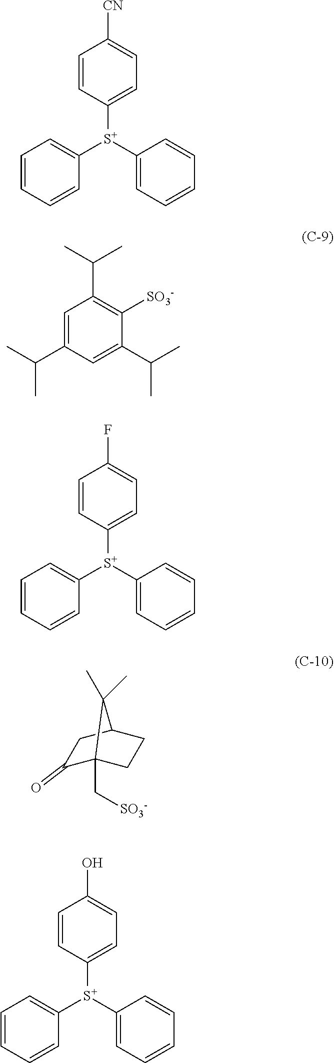

(D) Acid Diffusion Control Agent

The compounds (D-1) to (D-14) and compounds (hereinafter, may be also referred to as “compounds (d-1) to (d-2)”) represented by the following formula (d-1) to (d-2) were used as the acid diffusion control agent (D).

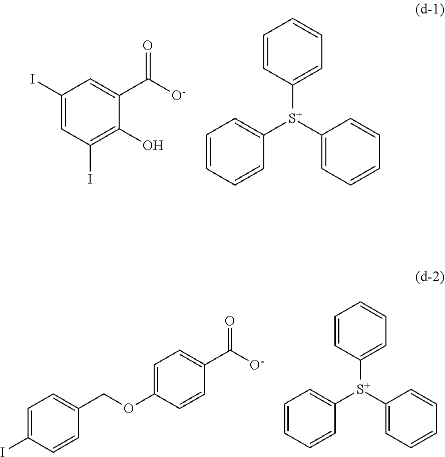 [SMILES: O=C([O-])c1cc(I)cc(I)c1O ; c1ccc([S+](c2ccccc2)c2ccccc2)cc1 ; O=C([O-])c1ccc(OCc2ccc(I)cc2)cc1 ; c1ccc([S+](c2ccccc2)c2ccccc2)cc1]

(E) Organic Solvent

The following (E-1) and (E-2) were used as the organic solvent (E).

(E-1): propylene glycol monomethyl ether acetate

(E-2): propylene glycol monomethyl ether

### Example 1: Preparation of Radiation-Sensitive Resin Composition (R-1)

100 parts by mass of (A-1) as the polymer (A), 1 part by mass of (B-1) as the polymer (B), 22 parts by mass of (C-1) as the acid generating agent (C), (D-1) as the acid diffusion control agent in an amount of 20 mol % with respect to (C-1), and 5,500 parts by mass of (E-1) and 1,500 parts by mass of (E-2) as the organic solvent (E) were admixed. A mix liquid thus obtained was filtered through a membrane filter having a pore size of 0.20 μm, whereby a radiation-sensitive resin composition (R-1) was prepared.

### Examples 2 to 30 and Comparative Examples 1 to 2: Preparation of Radiation-Sensitive Resin Composition (R-2) to (R-30) and (CR-1) to (CR-2)

Similarly to Example 1 except that each component of the following type and in the following content shown in Table 2 below was used, radiation-sensitive resin compositions (R-2) to (R-30) and (CR-1) to (CR-2) were prepared.

TABLE 2

| | | | | | | (C) Acid | | (D) Acid diffusion | | | |
|---|---|---|---|---|---|---|---|---|---|---|---|
| | Radiation- | (A) Polymer | | (B) Polymer | | generating agent | | control agent | | (E) Solvent | |
| | sensitive | | content | | content | | content | | content | | content |
| | resin | | (parts | | (parts | | (parts | | (% | | (parts by |
| | composition | type | by mass) | type | by mass) | type | by mass) | type | by mole) | type | mass) |
| Example 1 | R-1 | A-1 | 100 | B-1 | 1 | C-1 | 22 | D-1 | 20 | E-1/E-2 | 5,500/1,500 |
| Example 2 | R-2 | A-1 | 100 | B-1 | 1 | C-1 | 22 | D-2 | 20 | E-1/E-2 | 5,500/1,500 |
| Example 3 | R-3 | A-1 | 100 | B-1 | 1 | C-1 | 22 | D-3 | 20 | E-1/E-2 | 5,500/1,500 |
| Example 4 | R-4 | A-1 | 100 | B-1 | 1 | C-1 | 22 | D-4 | 20 | E-1/E-2 | 5,500/1,500 |
| Example 5 | R-5 | A-1 | 100 | B-1 | 1 | C-1 | 22 | D-5 | 20 | E-1/E-2 | 5,500/1,500 |
| Example 6 | R-6 | A-1 | 100 | B-1 | 1 | C-1 | 22 | D-6 | 20 | E-1/E-2 | 5,500/1,500 |
| Example 7 | R-7 | A-1 | 100 | B-1 | 1 | C-1 | 22 | D-7 | 20 | E-1/E-2 | 5,500/1,500 |
| Example 8 | R-8 | A-1 | 100 | B-1 | 1 | C-1 | 22 | D-8 | 20 | E-1/E-2 | 5,500/1,500 |
| Example 9 | R-9 | A-1 | 100 | B-1 | 1 | C-1 | 22 | D-9 | 20 | E-1/E-2 | 5,500/1,500 |
| Example 10 | R-10 | A-1 | 100 | B-1 | 1 | C-1 | 22 | D-10 | 20 | E-1/E-2 | 5,500/1,500 |
| Example 11 | R-11 | A-1 | 100 | B-1 | 1 | C-1 | 22 | D-11 | 20 | E-1/E-2 | 5,500/1,500 |
| Example 12 | R-12 | A-1 | 100 | B-1 | 1 | C-1 | 22 | D-12 | 20 | E-1/E-2 | 5,500/1,500 |
| Example 13 | R-13 | A-1 | 100 | B-1 | 1 | C-1 | 22 | D-13 | 20 | E-1/E-2 | 5,500/1,500 |
| Example 14 | R-14 | A-1 | 100 | B-1 | 1 | C-1 | 22 | D-14 | 20 | E-1/E-2 | 5,500/1,500 |
| Example 15 | R-15 | A-2 | 100 | B-1 | 1 | C1 | 22 | D-4 | 20 | E-1/E-2 | 5,500/1,500 |
| Example 16 | R-16 | A-3 | 100 | B-1 | 1 | C-1 | 22 | D-4 | 20 | E-1/E-2 | 5,500/1,500 |
| Example 17 | R-17 | A-4 | 100 | B-1 | 1 | C-1 | 22 | D-4 | 20 | E-1/E-2 | 5,500/1.500 |
| Example 18 | R-18 | A-5 | 100 | B-1 | 1 | C-1 | 22 | D-4 | 20 | E-1/E-2 | 5,500/1.500 |
| Example 19 | R-19 | A-6 | 100 | B-1 | 1 | C-1 | 22 | D-4 | 20 | E-1/E-2 | 5,500/1.500 |
| Example 20 | R-20 | A-7 | 100 | B-1 | 1 | C-1 | 22 | D-4 | 20 | E-1/E-2 | 5,500/1.500 |
| Example 21 | R-21 | A-8 | 100 | B-1 | 1 | C-1 | 22 | D-4 | 20 | E-1/E-2 | 5,500/1,500 |
| Example 22 | R-22 | A-1 | 100 | B-1 | 1 | C-2 | 22 | D-4 | 20 | E-1/E-2 | 5,500/1,500 |
| Example 23 | R-23 | A-1 | 100 | B-1 | 1 | C-3 | 22 | D-4 | 20 | E-1/E-2 | 5,500/1,500 |
| Example 24 | R-24 | A-1 | 100 | B-1 | 1 | C-4 | 22 | D-4 | 20 | E-1/E-2 | 5,500/1.500 |
| Example 25 | R-25 | A-1 | 100 | B-1 | 1 | C-5 | 22 | D-4 | 20 | E-1/E-2 | 5,500/1.500 |
| Example 26 | R-26 | A-1 | 100 | B-1 | 1 | C-6 | 22 | D-4 | 20 | E-1/E-2 | 5,500/1,500 |
| Example 27 | R-27 | A-1 | 100 | B-1 | 1 | C-7 | 22 | D-4 | 20 | E-1/E-2 | 5,500/1,500 |
| Example 28 | R-28 | A-1 | 100 | B-1 | 1 | C-8 | 22 | D-4 | 20 | E-1/E-2 | 5,500/1,500 |
| Example 29 | R-29 | A-1 | 100 | B-1 | 1 | C-9 | 22 | D-4 | 20 | E-1/E-2 | 5,500/1,500 |
| Example 30 | R-30 | A-1 | 100 | B-1 | 1 | C-10 | 22 | D-4 | 20 | E-1/E-2 | 5,500/1,500 |
| Comparative | CR-1 | A-1 | 100 | B-1 | 1 | C-1 | 22 | d-1 | 20 | E-1/E-2 | 5,500/1,500 |
| Example 1 | | | | | | | | | | | |
| Comparative | CR-2 | A-1 | 100 | B-1 | 1 | C-1 | 22 | d-2 | 20 | E-1/E-2 | 5,500/1,500 |
| Example 2 | | | | | | | | | | | |

Formation of Resist Pattern

By using a spin coater (“CLEAN TRACK ACT 12,” available from Tokyo Electron Limited), each radiation-sensitive resin composition prepared as described above was applied on a 12-inch silicon wafer surface provided with an underlayer film (“AL412” available from Brewer Science, Inc.) having an average thickness of 20 nm formed thereon. A resist film having an average thickness of 30 nm was formed through prebaking (PB) carried out at 130° C. for 60 sec, followed by cooling at 23° C. for 30 sec. Next, this resist film was irradiated with EUV light by using an EUV scanner (“NXE3300” available from ASML Co.: NA=0.33, irradiation conditions: Conventional s=0.89, maskimecDEFECT32FFR02). After the irradiation, the resist film was subjected to post exposure baking (PEB) at 130° C. for 60 sec. Subsequently, development was performed using a 2.38% by mass aqueous TMAH solution at 23° C. for 30 sec to form a positive-tone contact hole pattern (diameter: 25 nm; 50 nm pitch).

Evaluations

Each resist pattern formed as described above was evaluated on the sensitivity, the CDU performance, and the resolution in accordance with the following methods. Line-width measurement of the resist pattern was performed using a scanning electron microscope (“CG-4100” available from Hitachi High-Tech Corporation). The results of the evaluations are shown in Table 3 below.

Sensitivity

An exposure dose at which a contact hole pattern with a diameter of 25 nm was formed in the aforementioned resist pattern formation was defined as an optimum exposure dose, and this optimum exposure dose was adopted as Eop (mJ/cm²). The sensitivity was assessed to be: “favorable” in a case of the Eop being no greater than 60 mJ/cm²; and “unfavorable” in a case of the Eop being greater than 60 mJ/cm².

CDU performance

The resist pattern was observed from above using the scanning electron microscope, and diameters on the contact hole pattern were measured at 800 sites in total at arbitrary locations to determine a 3 Sigma value from distribution of the measurement values and defined as “CDU” (unit: nm). The CDU value being smaller indicates more favorable CDU performance, revealing less variance of the hole diameters in greater ranges. The CDU performance was evaluated to be: “favorable” in a case of CDU being no greater than 4.5 nm; and “unfavorable” in a case of CDU being greater than 4.5 nm.

Resolution

A minimum diameter on the contact hole pattern resolved with varying exposure doses was measured in the aforementioned resist pattern formation, and the measurement value was adopted as resolution (unit: nm). The resolution value being smaller indicates more favorable resolution. The resolution was evaluated to be: “favorable” in a case of the resolution being no greater than 22 nm; and “unfavorable” in a case of the resolution being greater than 22 nm.

TABLE 3

| | Radiation-sensitive | Eop | CDU | Resolution |
|---|---|---|---|---|
| | resin composition | (mJ/cm²) | (nm) | (nm) |
| Example 1 | R-1 | 55 | 4.2 | 22 |
| Example 2 | R-2 | 58 | 4.3 | 21 |
| Example 3 | R-3 | 52 | 3.7 | 19 |
| Example 4 | R-4 | 45 | 3.9 | 19 |
| Example 5 | R-5 | 48 | 4.2 | 18 |
| Example 6 | R-6 | 55 | 3.7 | 18 |
| Example 7 | R-7 | 57 | 3.8 | 19 |
| Example 8 | R-8 | 53 | 3.9 | 20 |
| Example 9 | R-9 | 48 | 4.1 | 18 |
| Example 10 | R-10 | 47 | 4.1 | 19 |
| Example 11 | R-11 | 40 | 3.6 | 17 |
| Example 12 | R-12 | 43 | 4.0 | 19 |
| Example 13 | R-13 | 49 | 3.9 | 18 |
| Example 14 | R-14 | 50 | 3.9 | 19 |
| Example 15 | R-15 | 51 | 3.9 | 19 |
| Example 16 | R-16 | 53 | 3.7 | 20 |
| Example 17 | R-17 | 47 | 4.0 | 20 |
| Example 18 | R-18 | 57 | 3.7 | 18 |
| Example 19 | R-19 | 49 | 3.9 | 19 |
| Example 20 | R-20 | 46 | 4.0 | 18 |
| Example 21 | R-21 | 50 | 3.9 | 18 |
| Example 22 | R-22 | 53 | 3.8 | 19 |
| Example 23 | R-23 | 51 | 3.9 | 20 |
| Example 24 | R-24 | 49 | 3.9 | 19 |
| Example 25 | R-25 | 50 | 3.8 | 20 |
| Example 26 | R-26 | 48 | 4.0 | 20 |
| Example 27 | R-27 | 52 | 4.0 | 18 |
| Example 28 | R-28 | 44 | 4.1 | 18 |
| Example 29 | R-29 | 48 | 4.2 | 18 |
| Example 30 | R-30 | 50 | 4.0 | 19 |
| Comparative | CR-1 | 65 | 5.0 | 25 |
| Example 1 | | | | |
| Comparative | CR-2 | 83 | 6.2 | 23 |
| Example 2 | | | | |

Obviously, numerous modifications and variations of the present invention(s) are possible in light of the above teachings. It is therefore to be understood that within the scope of the appended claims, the invention may be practiced otherwise than as specifically described herein.

What is claimed is:

1. A radiation-sensitive resin composition comprising:
a polymer, solubility of which in a developer solution is capable of being altered by an action of an acid;
a radiation-sensitive acid generator; and
a compound represented by formula (1): 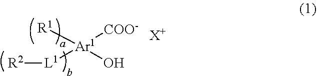
wherein, in the formula (1),
Ar¹ represents a group obtained by removing (a+b+2) hydrogen atoms from an aromatic hydrocarbon ring having 6 to 30 ring atoms;
R¹ represents a halogen atom or a monovalent organic group having 1 to 20 carbon atoms;
L¹ represents a divalent linking group;
R² represents a substituted or unsubstituted monovalent aromatic hydrocarbon group having 6 to 20 carbon atoms;
a is an integer of 0 to 10; b is an integer of 1 to 10, wherein a sum of a and b is no greater than 10, and wherein in a case in which a is no less than 2, a plurality of R¹s are identical or different from each other, and wherein in a case in which b is no less than 2, a plurality of L¹s are identical or different from each other, and a plurality of R²s are identical or different from each other; and
X⁺ represents a monovalent radiation-sensitive onium cation.

2. The radiation-sensitive resin composition according to claim 1, wherein R² in the formula (1) represents a monovalent aromatic hydrocarbon group having 6 to 20 carbon atoms in which at least one hydrogen atom on an aromatic ring is substituted with an iodine atom.

3. The radiation-sensitive resin composition according to claim 1, wherein L¹ in the formula (1) represents a carbonyloxy group, an oxycarbonyl group, an ether group, a sulfide group, an alkanediyl group having 1 to 5 carbon atoms, or a group being a combination thereof.

4. The radiation-sensitive resin composition according to claim 1, wherein the carboxylate group and the hydroxy group in the formula (1) bond to carbon atoms constituting Ar¹, respectively, the carbon atom to which the carboxylate group bonds and the carbon atom to which the hydroxy group bonds are directly bonded to each other.

5. The radiation-sensitive resin composition according to claim 1, wherein the polymer comprises a structural unit comprising an acid-labile group.

6. The radiation-sensitive resin composition according to claim 5, wherein the structural unit is represented by formula (2-1) or (2-2): 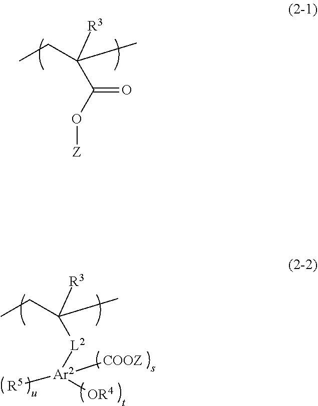
wherein,
in the formulae (2-1) and (2-2), R³ represents a hydrogen atom, a fluorine atom, a methyl group, or a trifluoromethyl group; and
Z represents an acid-labile group, and
in the formula (2-2),
L² represents a single bond, —COO—, —CONH—, or —O—;
Ar² represents a group obtained by removing (s+t+u+1) hydrogen atoms from an aromatic hydrocarbon ring having 6 to 20 ring atoms;
s is an integer of 0 to 10; t is an integer of 0 to 10, wherein a sum of s and t is an integer of 1 to 10, and wherein in a case in which s is no less than 2, a plurality of Zs are identical or different from each other, in a case in which s is no less than 1, R⁴ represents a hydrogen atom or a monovalent organic group having 1 to 20 carbon atoms, and wherein in a case in which t is no less than 2, a plurality of R⁴s are identical or different from each other, in a case in which s is 0 and t is 1, R⁴ represents an acid-labile group, and wherein in a case in which s is 0 and t is no less than 2, at least one of a plurality of R⁴s represents an acid-labile group;
R⁵ represents a halogen atom or a monovalent organic group having 1 to 20 carbon atoms; and
u is an integer of 0 to 10, wherein in a case in which u is no less than 2, a plurality of R⁵s are identical or different from each other, and wherein a sum of s, t, and u is no greater than 10.

7. A method of forming a resist pattern, the method comprising:
forming a resist film directly or indirectly on a substrate by applying the radiation-sensitive resin composition according to claim 1;
exposing the resist film; and
developing the resist film exposed.

8. The method according to claim 7, wherein R² in the formula (1) represents a monovalent aromatic hydrocarbon group having 6 to 20 carbon atoms in which at least one hydrogen atom on an aromatic ring is substituted with an iodine atom.

9. The method according to claim 7, wherein L¹ in the formula (1) represents a carbonyloxy group, an oxycarbonyl group, an ether group, a sulfide group, an alkanediyl group having 1 to 5 carbon atoms, or a group being a combination thereof.

10. The method according to claim 7, wherein the carboxylate group and the hydroxy group in the formula (1) bond to carbon atoms constituting Ar¹, respectively, the carbon atom to which the carboxylate group bonds and the carbon atom to which the hydroxy group bonds are directly bonded to each other.

11. The method according to claim 7, wherein the polymer comprises a structural unit comprising an acid-labile group.

12. The method according to claim 11, wherein the structural unit is represented by formula (2-1) or (2-2): 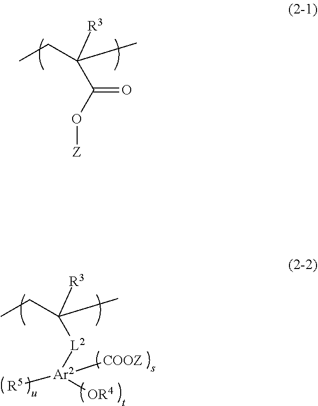
wherein,
in the formulae (2-1) and (2-2), R³ represents a hydrogen atom, a fluorine atom, a methyl group, or a trifluoromethyl group; and
Z represents an acid-labile group, and
in the formula (2-2),
L² represents a single bond, —COO—, —CONH—, or —O—;
Ar² represents a group obtained by removing (s+t+u+1) hydrogen atoms from an aromatic hydrocarbon ring having 6 to 20 ring atoms;
s is an integer of 0 to 10; t is an integer of 0 to 10, wherein a sum of s and t is an integer of 1 to 10, and wherein in a case in which s is no less than 2, a plurality of Zs are identical or different from each other, in a case in which s is no less than 1, R⁴ represents a hydrogen atom or a monovalent organic group having 1 to 20 carbon atoms, and wherein in a case in which t is no less than 2, a plurality of R⁴s are identical or different from each other, in a case in which s is 0 and t is 1, R⁴ represents an acid-labile group, and wherein in a case in which s is 0 and t is no less than 2, at least one of a plurality of R⁴s represents an acid-labile group;
R⁵ represents a halogen atom or a monovalent organic group having 1 to 20 carbon atoms; and
u is an integer of 0 to 10, wherein in a case in which u is no less than 2, a plurality of R⁵s are identical or different from each other, and wherein a sum of s, t, and u is no greater than 10.

13. A compound represented by formula (1): 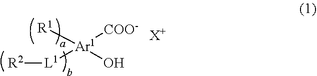
wherein, in the formula (1),
Ar¹ represents a group obtained by removing (a+b+2) hydrogen atoms from an aromatic hydrocarbon ring having 6 to 30 ring atoms;
R¹ represents a halogen atom or a monovalent organic group having 1 to 20 carbon atoms;
L¹ represents a divalent linking group;
R² represents a substituted or unsubstituted monovalent aromatic hydrocarbon group having 6 to 20 carbon atoms;
a is an integer of 0 to 10; b is an integer of 1 to 10, wherein a sum of a and b is no greater than 10, and wherein in a case in which a is no less than 2, a plurality of R¹s are identical or different from each other, and wherein in a case in which b is no less than 2, a plurality of Lis are identical or different from each other, and a plurality of R²s are identical or different from each other; and
X⁺ represents a monovalent radiation-sensitive onium cation.
